SEQUENCE LISTING (55 sequences)

SEQ ID NO: 1 - RNA, 24 nt, Synthetic construct
  source 1..24: mol_type = other RNA
  ncRNA 1..24: ncRNA_class = antisense_RNA
ctgaaatacatatataaaaaggtc

SEQ ID NO: 2 - RNA, 24 nt, Synthetic construct
  source 1..24: mol_type = other RNA
  ncRNA 1..24: ncRNA_class = antisense_RNA
ctgaaatacatatataagaaggtc

SEQ ID NO: 3 - RNA, 25 nt, Synthetic construct
  source 1..25: mol_type = other RNA
  ncRNA 1..25: ncRNA_class = antisense_RNA
ctgaaatacatatataagaaggtca

SEQ ID NO: 4 - RNA, 25 nt, Synthetic construct
  source 1..25: mol_type = other RNA
  ncRNA 1..25: ncRNA_class = antisense_RNA
tacatatataagaaggtcattgtaa

SEQ ID NO: 5 - RNA, 25 nt, Synthetic construct
  source 1..25: mol_type = other RNA
  ncRNA 1..25: ncRNA_class = antisense_RNA
aaaggtcattgtaagaatttctatg

SEQ ID NO: 6 - RNA, 25 nt, Synthetic construct
  source 1..25: mol_type = other RNA
  ncRNA 1..25: ncRNA_class = antisense_RNA
gtaaaaatttctatgtatgtgaaca

SEQ ID NO: 7 - RNA, 25 nt, Synthetic construct
  source 1..25: mol_type = other RNA
  ncRNA 1..25: ncRNA_class = antisense_RNA
tctatgtatgtgaacatatccatag

SEQ ID NO: 8 - RNA, 25 nt, Synthetic construct
  source 1..25: mol_type = other RNA
  ncRNA 1..25: ncRNA_class = antisense_RNA
aatcttctgtctctgcttgagttct

SEQ ID NO: 9 - RNA, 17 nt, Synthetic construct
  source 1..17: mol_type = other RNA
  ncRNA 1..17: ncRNA_class = antisense_RNA
ctctgcttgagttcttt

SEQ ID NO: 10 - RNA, 17 nt, Synthetic construct
  source 1..17: mol_type = other RNA
  ncRNA 1..17: ncRNA_class = antisense_RNA
tgtctctgcttgagttc

SEQ ID NO: 11 - RNA, 17 nt, Synthetic construct
  source 1..17: mol_type = other RNA
  ncRNA 1..17: ncRNA_class = antisense_RNA
ttctgtctctgcttgag

SEQ ID NO: 12 - RNA, 17 nt, Synthetic construct
  source 1..17: mol_type = other RNA
  ncRNA 1..17: ncRNA_class = antisense_RNA
atcttctgtctctgctt

SEQ ID NO: 13 - RNA, 17 nt, Synthetic construct
  source 1..17: mol_type = other RNA
  ncRNA 1..17: ncRNA_class = antisense_RNA
ctaatcttctgtctctg

SEQ ID NO: 14 - RNA, 17 nt, Synthetic construct
  source 1..17: mol_type = other RNA
  ncRNA 1..17: ncRNA_class = antisense_RNA
tgaacatatccatagga

SEQ ID NO: 15 - RNA, 17 nt, Synthetic construct
  source 1..17: mol_type = other RNA
  ncRNA 1..17: ncRNA_class = antisense_RNA
atgtgaacatatccata

SEQ ID NO: 16 - RNA, 17 nt, Synthetic construct
  source 1..17: mol_type = other RNA
  ncRNA 1..17: ncRNA_class = antisense_RNA
tgtatgtgaacatatcc

SEQ ID NO: 17 - RNA, 17 nt, Synthetic construct
  source 1..17: mol_type = other RNA
  ncRNA 1..17: ncRNA_class = antisense_RNA
ctatgtatgtgaacata

SEQ ID NO: 18 - RNA, 17 nt, Synthetic construct
  source 1..17: mol_type = other RNA
  ncRNA 1..17: ncRNA_class = antisense_RNA
tttctatgtatgtgaac

SEQ ID NO: 19 - RNA, 17 nt, Synthetic construct
  source 1..17: mol_type = other RNA
  ncRNA 1..17: ncRNA_class = antisense_RNA
aaatttctatgtatgtg

SEQ ID NO: 20 - RNA, 17 nt, Synthetic construct
  source 1..17: mol_type = other RNA
  ncRNA 1..17: ncRNA_class = antisense_RNA
taaaaatttctatgtat

SEQ ID NO: 21 - RNA, 17 nt, Synthetic construct
  source 1..17: mol_type = other RNA
  ncRNA 1..17: ncRNA_class = antisense_RNA
ttgtaaaaatttctatg

SEQ ID NO: 22 - RNA, 17 nt, Synthetic construct
  source 1..17: mol_type = other RNA
  ncRNA 1..17: ncRNA_class = antisense_RNA
tcattgtaaaaatttct

SEQ ID NO: 23 - RNA, 17 nt, Synthetic construct
  source 1..17: mol_type = other RNA
  ncRNA 1..17: ncRNA_class = antisense_RNA
aggtcattgtaaaaatt

SEQ ID NO: 24 - RNA, 17 nt, Synthetic construct
  source 1..17: mol_type = other RNA
  ncRNA 1..17: ncRNA_class = antisense_RNA
aaaaggtcattgtaaaa

SEQ ID NO: 25 - RNA, 17 nt, Synthetic construct
  source 1..17: mol_type = other RNA
  ncRNA 1..17: ncRNA_class = antisense_RNA
ataaaaaggtcattgta

SEQ ID NO: 26 - RNA, 17 nt, Synthetic construct
  source 1..17: mol_type = other RNA
  ncRNA 1..17: ncRNA_class = antisense_RNA
tatataaaaaggtcatt

SEQ ID NO: 27 - RNA, 17 nt, Synthetic construct
  source 1..17: mol_type = other RNA
  ncRNA 1..17: ncRNA_class = antisense_RNA
acatatataaaaaggtc

SEQ ID NO: 28 - RNA, 17 nt, Synthetic construct
  source 1..17: mol_type = other RNA
  ncRNA 1..17: ncRNA_class = antisense_RNA
aatacatatataaaaag

SEQ ID NO: 29 - RNA, 17 nt, Synthetic construct
  source 1..17: mol_type = other RNA
  ncRNA 1..17: ncRNA_class = antisense_RNA
tgaaatacatatataaa

SEQ ID NO: 30 - RNA, 17 nt, Synthetic construct
  source 1..17: mol_type = other RNA
  ncRNA 1..17: ncRNA_class = antisense_RNA
ttctgaaatacatatat

SEQ ID NO: 31 - RNA, 17 nt, Synthetic construct
  source 1..17: mol_type = other RNA
  ncRNA 1..17: ncRNA_class = antisense_RNA
aaattctgaaatacata

SEQ ID NO: 32 - DNA, 2414 bp, Synthetic construct
  source 1..2414: mol_type = other DNA
  intron 1..2296: note = OPA1 intron 7
  exon 2297..2414: note = OPA1 exon7X
gtgatggatggtttaagggggctaccgatacattcacactaatcagccatttctgccaagatcatgtcacctcaatctgttcatggactccaaatacaagaaattaatttgacaaagtgaaaatataaaagatgcatcatataaatatgtaacttttctggagtgggtagtataggtaaagccaaaagaaacaaattcaagcagaggaattttggtttctgaaaattaggttgtctgtagggtccctgtatttatacttagaacaaaattaggaatttctgtttatgtggtccagttattgagtcaccctaagtttgtaggcatcttacctacctacttgctccccaagtttttatttctaaaatgaaaagcattgctgtagatgaccagtttacactaaagaataacatttatttatttgttttagctaaagtatatggacagggaacattcatattcttgtagaagaaaattattttgacttttgggcaaaagcatgtagttcttatacactttgacaaactcattgcgtacatttttcacattaatcaaagtcagcacaaataaattttcaccttggaccacggagggtttgaacactggaaatttgatataattctggttgctaaagaacaagttctaataaaagcttaagtgtataccaatatgtggctgttggtgcaatcagcaggtccgtaaaaatatgattttaatggttaggtaatcccacaacggagatcccaaagttcatgtttggaagagacttttgggtcaaagtgaaatcagtgtaatgaatttaaaattatactctgagatcttgaaatcagctaattatgttacatcttattagctcagaaaagttttgaagttatatacaaatgctagtcaggaaaaaagattcagtcatgtaattcttgtacattctactatttaaatcaaccaatattatagattatgatttagtgcagtaattctgctggctaaccttatctcatttggtggtggttagtacttcagagtactcaccatagtttcatttatgttttcagcatcacttcctggtttttctcaattccatggctgtggaatcaattcatatgtatatttagcttcggtgagcaaaaacatagctagaaaaagaaaagaagtgagtttcctacctggttaaattaaagtcgatgtgttaagccaaggaggacttcttttgaatggtactttaacaatccctgttctgtatactgtgaatatatcatttaaatagcctaataaattggatgcttaggctgagccacctatactttagttttgttatggaaagaagggagaggagcaagtatgttcttatatgttacttagaaataagaatgtagctgtagttacacattgttcttaagtttttttcgtaagacaacttgaaatgagtcccataggcctgctatttaacattctaagatatgacttaaggttaatgatgagcttttgaatctgacaattcaagagatatccataatgaatactgattcattttctacattgctgaaagctaatgttcattttaagcctactttagtagcctttatttgggcttagagatgttattcctctttctgatatttattgggttatctgtttaacccttttatatctccctttcccgatttgtaaattagagactggcaagactttttaccctgagtagagcaccaaacatggcttgtttctgcccacactgtagttaccttgaggggaagtaaatgggactttaaaagcaatttatgctcttttatagtgaaattatccctcttactatcccgaaagactgttaccttacaatatcctccactcctttccccctgtagttactatagagatgacttttcggttcttcactgccataatgatcaaaatcctaattcatgagatttttatcattccaggcatgtgaggtttacttgatgcataaaaccgcaagtactttttgttgttttttaattgttttttctctcttatcttcttgaaagtctaagtagatcatcatttttgatgtcttattagtagcaactaataaattttccctgtatcttctcagcaaaagaactcaagcagagacagaagattagaactaccattggtagttttgcttcctatggatatgttcacatacatagaaatttttacaatgacctttttatatatgtatttcagaatttcagaatggcctcaatgccttaataggaagaaatacttgaaatttttaaattagggcttggttttgtgaggagctagtaaaggtttttctctttcagctttagcttgtttctgcggaggattccgctctttctccatcagtttcatagccctggaattgtagaaaagctctggtttcaagaccattgatatccatttctgtcagg

SEQ ID NO: 33 - RNA, 25 nt, Synthetic construct
  source 1..25: mol_type = other RNA
  ncRNA 1..25: ncRNA_class = antisense_RNA
ctgaaatacatatataaaaaggtca

SEQ ID NO: 34 - RNA, 26 nt, Synthetic construct
  source 1..26: mol_type = other RNA
  ncRNA 1..26: ncRNA_class = antisense_RNA
ctgaaatacatatataaaaaggtcat

SEQ ID NO: 35 - RNA, 27 nt, Synthetic construct
  source 1..27: mol_type = other RNA
  ncRNA 1..27: ncRNA_class = antisense_RNA
ctgaaatacatatataaaaaggtcatt

SEQ ID NO: 36 - RNA, 28 nt, Synthetic construct
  source 1..28: mol_type = other RNA
  ncRNA 1..28: ncRNA_class = antisense_RNA
ctgaaatacatatataaaaaggtcattg

SEQ ID NO: 37 - RNA, 29 nt, Synthetic construct
  source 1..29: mol_type = other RNA
  ncRNA 1..29: ncRNA_class = antisense_RNA
ctgaaatacatatataaaaaggtcattgt

SEQ ID NO: 38 - RNA, 30 nt, Synthetic construct
  source 1..30: mol_type = other RNA
  ncRNA 1..30: ncRNA_class = antisense_RNA
ctgaaatacatatataaaaaggtcattgta

SEQ ID NO: 39 - RNA, 30 nt, Synthetic construct
  source 1..30: mol_type = other RNA
  ncRNA 1..30: ncRNA_class = antisense_RNA
tgaaatacatatataaaaaggtcattgtaa

SEQ ID NO: 40 - RNA, 30 nt, Synthetic construct
  source 1..30: mol_type = other RNA
  ncRNA 1..30: ncRNA_class = antisense_RNA
gaaatacatatataaaaaggtcattgtaaa

SEQ ID NO: 41 - RNA, 30 nt, Synthetic construct
  source 1..30: mol_type = other RNA
  ncRNA 1..30: ncRNA_class = antisense_RNA
aaatacatatataaaaaggtcattgtaaaa

SEQ ID NO: 42 - RNA, 30 nt, Synthetic construct
  source 1..30: mol_type = other RNA
  ncRNA 1..30: ncRNA_class = antisense_RNA
aatacatatataaaaaggtcattgtaaaaa

SEQ ID NO: 43 - RNA, 30 nt, Synthetic construct
  source 1..30: mol_type = other RNA
  ncRNA 1..30: ncRNA_class = antisense_RNA
atacatatataaaaaggtcattgtaaaaat

SEQ ID NO: 44 - RNA, 30 nt, Synthetic construct
  source 1..30: mol_type = other RNA
  ncRNA 1..30: ncRNA_class = antisense_RNA
tacatatataaaaaggtcattgtaaaaatt

SEQ ID NO: 45 - RNA, 30 nt, Synthetic construct
  source 1..30: mol_type = other RNA
  ncRNA 1..30: ncRNA_class = antisense_RNA
acatatataaaaaggtcattgtaaaaattt

SEQ ID NO: 46 - RNA, 25 nt, Synthetic construct
  source 1..25: mol_type = other RNA
  ncRNA 1..25: ncRNA_class = antisense_RNA
ctgaaatacatatataaaaaggtca

SEQ ID NO: 47 - RNA, 25 nt, Synthetic construct
  source 1..25: mol_type = other RNA
  ncRNA 1..25: ncRNA_class = antisense_RNA
tacatatataaaaaggtcattgtaa

SEQ ID NO: 48 - RNA, 30 nt, Synthetic construct; identical to SEQ ID NO: 38
  source 1..30: mol_type = other RNA
  ncRNA 1..30: ncRNA_class = antisense_RNA

SEQ ID NO: 49 - RNA, 30 nt, Synthetic construct; identical to SEQ ID NO: 39
  source 1..30: mol_type = other RNA
  ncRNA 1..30: ncRNA_class = antisense_RNA

SEQ ID NO: 50 - RNA, 30 nt, Synthetic construct; identical to SEQ ID NO: 40
  source 1..30: mol_type = other RNA
  ncRNA 1..30: ncRNA_class = antisense_RNA

SEQ ID NO: 51 - RNA, 30 nt, Synthetic construct; identical to SEQ ID NO: 41
  source 1..30: mol_type = other RNA
  ncRNA 1..30: ncRNA_class = antisense_RNA

SEQ ID NO: 52 - RNA, 30 nt, Synthetic construct
  source 1..30: mol_type = other RNA
  ncRNA 1..30: ncRNA_class = antisense_RNA
gaaattctgaaatacatatataaaaaggtc

SEQ ID NO: 53 - RNA, 30 nt, Synthetic construct
  source 1..30: mol_type = other RNA
  ncRNA 1..30: ncRNA_class = antisense_RNA
aaattctgaaatacatatataaaaaggtca

SEQ ID NO: 54 - RNA, 30 nt, Synthetic construct
  source 1..30: mol_type = other RNA
  ncRNA 1..30: ncRNA_class = antisense_RNA
aattctgaaatacatatataaaaaggtcat

SEQ ID NO: 55 - protein, 30 aa, Synthetic construct
RRSRTARAGRPGRNSSRPSAPRGASGGASG

(19) United States
(12) Patent Application Publication
(10) Pub. No.: US 2025/0002917 A1
(43) Pub. Date: Jan. 2, 2025

(54) METHOD OF TREATMENT FOR OPTIC ATROPHY

(71) Applicant: PYC Therapeutics Limited, Nedlands, Victoria (AU)

(72) Inventors: Janya Grainok, Nedlands (AU); Kim Rice, Nedlands (AU); Sasiwimon Utama, Nedlands (AU)

(21) Appl. No.: 18/834,170

(22) PCT Filed: Jan. 31, 2023
(86) PCT No.: PCT/AU2023/050053

§ 371 Date: Jul. 29, 2024

(30) Foreign Application Priority Data
Jan. 31, 2022 (AU) 2022900163

(51) Int. Cl. C12N 15/113; A61P 27/02

(52) U.S. Cl. CPC C12N 15/113; A61P 27/02; C12N 2310/11; C12N 2310/321; C12N 2310/3233; C12N 2310/3513

(57) ABSTRACT

The present invention relates to isolated or purified antisense oligonucleotides that bind to intron 7 of an OPA1 gene pre-mRNA. The present invention also relates to methods of manipulating translation of the OPA1 gene transcript and use of the antisense oligonucleotide to treat. prevent or ameliorate the effects caused by mutations in the gene OPA1.

### RELATED APPLICATION DATA

The present application claims priority from Australian Patent Application No. 2022900163 filed on 31 Jan. 2022 entitled “Method of Treatment for Optic Atrophy”, the entire contents of which is hereby incorporated by reference.

### SEQUENCE LISTING

The present application is filed together with a Sequence Listing in electronic form. The entire contents of the Sequence Listing are hereby incorporated by reference.

### TECHNICAL FIELD The present invention relates to the use of antisense oligonucleotide to treat, prevent or ameliorate the effects caused by mutations in the gene OPA1.

### BACKGROUND

Autosomal dominant optic atrophy (ADOA) is the world's commonest hereditary optic neuropathy, with an estimated disease prevalence ranging from 1 in 10,000 to 1 in 50,000. OPA1 is the major causative gene in most families with ADOA.

ADOA classically presents in early childhood with progressive visual failure, with selective retinal ganglion cell loss as the defining pathological characteristic. The mean age of onset of visual failure is 7 years, with a range of 1 to 16 years. Eighty percent of affected individuals are symptomatic before the age of 10 years. In up to 20% of cases, ADOA is associated with other clinical manifestations, most commonly sensorineural deafness, ataxia, myopathy and progressive external ophthalmoplegia. The significance of these syndromal ADOA variants has been highlighted by the identification of cytochrome c oxidase (COX)-deficient muscle fibres and multiple mtDNA deletions in skeletal muscle biopsies from these patients, implicating a role of OPA 1 in mitochondrial DNA maintenance.

OPA1 consists of 30 exons spanning 100 kb of genomic DNA on the long arm of chromosome 3 (3q28-q29), and the protein product is a 960 amino acid polypeptide that co-localizes to the inner mitochondrial membrane. OPA1 contains a highly conserved functional GTPase domain shared by members of the dynamin superfamily of mechanoenzymes and regulates several important cellular processes including the stability of the mitochondrial network. Over 400 different OPA1 mutations have been reported to be responsible for optic nerve degeneration and visual loss, ranging from isolated ‘Dominant Optic Atrophy’ (DOA; OMIM #165500) to more severe multi-systemic syndromes named ‘DOAplus’ (OMIM #125250), including some bi-allelic cases with Behr Syndrome (OMIM #210000).

OPA1 is a ubiquitously expressed mitochondrial GTPase that is indispensable for mitochondrial function. In humans, OPA1 generates at least eight isoforms via differential splicing of exons 4, 4b and 5b or equivalent to exon 4, 5 and 7 according to FIG. 1A. OPA1 precursor proteins are targeted and mobilised to the mitochondria by their mitochondrial targeting sequence (MTS). In the mitochondria, the OPA1 precursor proteins are cleaved into either long forms (1 forms) that are anchored to the inner mitochondrial membrane, or into short, soluble forms (s forms).

The coding sequence of the full OPA1 gene is beyond the packaging capacity of AAV vectors, which have a limit of less than 5 kb. Furthermore, certain forms of CRISPR/Cas9 gene correction will require a different product for each unique OPA1 mutation, and splicing switching strategies using antisense oligomers targeted to the OPA1 pre-mRNA may only target regions of mutations (e.g., within an exon, as with antisense oligomer drugs like Eteplirsen) and may not provide a functional protein product. In addition, both the gene replacement and gene editing approaches require subretinal injection of viral vectors to achieve adequate transfection. The procedure carries risks of retinal trauma.

There is a need to provide new treatments or preventative measures for ADOA, or at least the provision of methods to compliment the previously known treatments. The present invention seeks to provide an improved or alternative method for treating, preventing or ameliorating the effects of ADOA.

The previous discussion of the background art is intended to facilitate an understanding of the present invention only. The discussion is not an acknowledgement or admission that any of the material referred to is or was part of the common general knowledge as at the priority date of the application.

SUMMARY OF INVENTION

The present invention provides a composition comprising an antisense oligonucleotide that binds to a targeted portion of a OPA1 gene pre-mRNA in a cell to promote exclusion of a nonsense-mediated RNA decay-inducing (NMD) exon during splicing of OPA1 pre-mRNA, to increase the level of OPA1 mRNA transcripts encoding OPA1 protein.

The invention provides a means to increase levels of functional OPA1 protein expression. Preferably the increased functional OPA1 is in patients who have ADOA, more preferably in ADOA where OPA1 haploinsufficiency is implicated.

Broadly, according to one aspect of the invention, there is provided an isolated or purified antisense oligonucleotide that binds to intron 7 of a OPA1 gene pre-mRNA in a cell to promote exclusion of nonsense-mediated RNA decay-inducing (NMD) exon 7x, derived from intron 7, during splicing of OPA1 pre-mRNA. Preferably, there is provided an isolated or purified antisense oligonucleotide to increase translation of functional OPA1 protein through exclusion of an NMD exon. Preferably, there is provided an isolated or purified antisense oligonucleotide for inducing increased production of functional OPA1 protein or part thereof.

In one aspect of the invention, there is provided an antisense oligonucleotide of 10 to 50 nucleotides comprising a targeting sequence complementary to a region near or within intron 7 or part thereof. In another aspect of the invention, there is provided an antisense oligonucleotide of 10 to 50 nucleotides comprising a targeting sequence complementary to a region near or within an exon of the OPA1 gene transcript or part thereof.

According to an aspect of the invention, there is provided an isolated or purified antisense oligonucleotide designed to increase translation of functional OPA1 protein through exclusion of nonsense-mediated RNA decay-inducing (NMD) exon 7x. Preferably, the antisense oligonucleotide is a phosphorodiamidate morpholino oligonucleotide.

Preferably, the antisense oligonucleotide is selected from the group comprising the sequences set forth in Table 1 and Table 2 and combinations or cocktails thereof.

More specifically, the antisense oligonucleotide may be selected from the group comprising of any one or more of SEQ ID Nos: 1-31; more preferably SEQ ID Nos: 1, 2, 3, 4 and 7; even more preferably SEQ ID Nos: 1, 3 and 4; and combinations or cocktails thereof.

In one example, the antisense oligonucleotide is selected from the group comprising of any one or more of SEQ ID Nos: 1-31.

In one example, the antisense oligonucleotide is selected from the group comprising of any one or more of SEQ ID Nos: 1, 2, 3, 4 and 7.

In one example, the antisense oligonucleotide is selected from the group comprising of any one or more of SEQ ID Nos: 1, 3 and 4.

Preferably, the antisense oligonucleotide is selected from the group comprising the sequences set forth in Tables 1, 2 and 4 and combinations or cocktails thereof.

More specifically, the antisense oligonucleotide may be selected from the group comprising of any one or more of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44; and combinations or cocktails thereof.

In one example, the antisense oligonucleotide is selected from the group comprising of any one or more of SEQ ID Nos: 1-31 and 33-54.

In one example, the antisense oligonucleotide is selected from the group comprising of any one or more of SEQ ID Nos: 33-54.

In one example, the antisense oligonucleotide is selected from the group comprising of any one or more of SEQ ID Nos: 1, 2, 3, 4, 7 and 33-44.

In one example, the antisense oligonucleotide is selected from the group comprising of any one or more of SEQ ID Nos: 33-44.

In one example, the antisense oligonucleotide is selected from the group comprising of any one or more of SEQ ID Nos: 1, 3, 4, 37, 40 and 44.

In one example, the antisense oligonucleotide is selected from the group comprising of any one or more of SEQ ID Nos: 37, 40 and 44. For example, the antisense oligonucleotide is set forth in SEQ ID No: 1. In another example, the antisense oligonucleotide is set forth in SEQ ID No: 3. In a further example, the antisense oligonucleotide is set forth in SEQ ID No: 4. For example, the antisense oligonucleotide is set forth in SEQ ID No: 37. In another example, the antisense oligonucleotide is set forth in SEQ ID No: 40. In a further example, the antisense oligonucleotide is set forth in SEQ ID No 44.

There is also provided a method for manipulating translation of the OPA1 gene transcript, the method including the step of:
a) providing one or more of the antisense oligonucleotides as described herein and allowing the oligonucleotide(s) to bind to a target nucleic acid site.

There is also provided a pharmaceutical, prophylactic, or therapeutic composition to treat, prevent or ameliorate the effects of a disease related to OPA1 expression in a patient, the composition comprising:
a) one or more antisense oligonucleotides as described herein and
b) one or more pharmaceutically acceptable carriers and/or diluents.

Preferably the disease associated with OPA1 expression is associated with decreased levels of functional OPA1 protein expression. Preferably the decreased functional OPA1 is in patients who have ADOA, more preferably in ADOA where OPA1 haploinsufficiency is implicated.

There is also provided a method to treat, prevent or ameliorate the effects of a disease associated with OPA1 expression, comprising the step of:
a) administering to the patient an effective amount of one or more antisense oligonucleotides or pharmaceutical composition comprising one or more antisense oligonucleotides as described herein.

There is also provided the use of purified and isolated antisense oligonucleotides as described herein, for the manufacture of a medicament to treat, prevent or ameliorate the effects of a disease associated with OPA1 expression.

There is also provided a kit to treat, prevent or ameliorate the effects of a disease associated with OPA1 expression in a patient, which kit comprises at least an antisense oligonucleotide as described herein and combinations or cocktails thereof, packaged in a suitable container, together with instructions for its use.

Preferably the disease associated with OPA1 expression in a patient is Autosomal Dominant Optic Atrophy (ADOA). The subject with the disease associated with OPA1 expression may be a mammal, including a human.

Further aspects of the invention will now be described with reference to the accompanying non-limiting examples and drawings.

### BRIEF DESCRIPTION OF THE DRAWINGS

FIG. 1 is a schematic of the antisense oligonucleotides targeting intron 7 of OPA1 to remove exon 7x. (A) Illustration of intron 7 and exon 7x in OPA1; SEQ ID NO 32. (B) Antisense oligonucleotides were designed to target the upstream region of OPA1 exon 7x (intron 7). PTC; Premature termination codon.

FIG. 2 shows screening of PMOs (25 and 50 μM) in ADOA patient fibroblasts. Patient fibroblasts were transfected for 48 hr with PMOs targeting removal of the OPA1 exon 7x as indicated. OPA1 transcript expression was assessed by digital droplet PCR (ddPCR) and normalised to GAPDH. RPL27 and SCL25A3 transcript levels. The OPA1 expression in untreated cells was set to 1.

FIG. 3 shows screening of PMOs (50 and 100 μM) in ADOA patient fibroblasts. (A) The western blot gel image shows expression of long and short OPA1 isoforms in patient fibroblasts transfected with PMOs targeting intron 7 of the OPA1 transcript at 48 hr. (B) The band intensity of OPA1 expression was normalised to beta-actin (assessed by ImageJ™). The OPA1 expression in untreated cells was set to 1.

FIG. 4 shows screening of cell penetrating peptide-conjugated PMOs (PPMOs) (5, 10 and 20 μM) in ADOA patient fibroblasts. ADOA patient fibroblasts were transfected for 5 days with PPMOs targeting intron 7 of the OPA1 transcript as indicated. OPA1 transcript expression was assessed by ddPCR and normalised to HPRT1. The OPA1 expression in untreated cells was set to 1.

FIG. 5 is a schematic of the refinement of antisense oligonucleotides to improve OPA1 upregulation. (A) Illustration of OPA1 exons and the location of exon 7x exists in the transcript. (B) Binding region of parental PMOs on OPA1 transcript upstream of exon7x. Exon 7x is not drawn to scale. (C) Binding region of daughter sequences with microwalk, nucleotide base substitution and lengthening to improve the efficacy of PMOs.

FIG. 6 shows screening of microwalked PMOs (25 and 50 μM) in ADOA patient fibroblasts. Patient fibroblasts were transfected in triplicates for 48 hr with PMOs targeting removal of the OPA1 exon 7x as indicated. Experiments were performed in 1-4 biological replicates as indicated with the number of data points within a bar graph. OPA1 transcript expression was assessed by ddPCR and normalised to the HPRT1 transcript level. The OPA1 expression in untreated cells was set to 1.

FIG. 7 shows the efficacy of the PMO OPA1_Ex7xA(−134−105)2 mmA>G (25 and 50 μM) for induction of OPA1 protein upregulation in ADOA patient fibroblasts. OPA1 protein expression was assessed using WB assay and the band intensity of OPA1 expression OPA1 was normalised to HPRT-1 (assessed by ImageJ™). The OPA1 expression in untreated cells was set to 1.

FIG. 8 shows the balance of major OPA1 isoforms with and without exon 7 in the transcripts following PMO treatment. Assessment of total OPA1 expression and OPA1 isoforms with (top portion of bar graph) and without exon 7 (bottom portion of bar graph) in PMO-treated and untreated fibroblasts derived from an ADOA patient. Total OPA1 transcript expression was assessed using ddPCR and normalised to the HPRT1 transcript level. The OPA1 expression in untreated cells was set to 1. The expression of OPA1 isoforms was analysed by RT-PCR following by automated capillary electrophoresis separation using a LabChip GX analyzer to quantify the band intensity of OPA1 isoforms.

FIG. 9 shows quantitative analysis of major OPA1 isoforms following PMO treatment. PMO OPA1_Ex7xA(−134−105)2 mmA>G (25 and 50 μM) was incubated to patient derived fibroblast and samples were harvested for quantification of total and OPA 1 isoform upregulation using a ddPCR assay. Primers targeting exons 22-23 and exons 6-8 were used to determine the levels of total OPA1 mRNA and OPA1 spliced isoforms without exon 7, respectively. Both total OPA1 mRNA and major OPA1 isoforms were normalised to the expression level of HPRT1. OPA1 expression in untreated cells was set to 1. Two-way ANOVA was used to test the statistical significance of data from 3 technical replicates. *p<0.05, **p<0.01.

FIG. 10 shows PPMO mediated total OPA1 mRNA upregulation in iPSC-RGCs derived from an ADOA patient. Peptide-conjugated PMO was incubated at concentrations of (5, 10 and 20 μM) to iPSC-RGCs for 120 hr and RNA was harvested to analyse the total OPA1 mRNA level. OPA1 transcript expression was assessed by ddPCR and normalised to the HPRT1 level. The OPA1 expression in untreated cells was set to 1. Two-way ANOVA was used to test the statistical significance of data from 3 technical replicates. **p<0.01, ****p<0.0001.

### DESCRIPTION OF INVENTION

### Detailed Description of the Invention

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such techniques are described and explained throughout the literature in sources such as Perbal 1984, Sambrook et al., 2001, Brown (editor) 1991, Glover and Hames (editors) 1995 and 1996, Ausubel et al. including all updates until present, Coligan et al. (editors) (including all updates until present), Maniatis et al. 1982, Gait (editor) 1984, Hames and Higgins (editors) 1984, Freshney (editor) 1986.

The term “and/or”, e.g. “X and/or Y” shall be understood to mean either “X and Y” or “X or Y” and shall be taken to provide explicit support for both meanings or for either meaning.

The term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “antisense oligonucleotide” “antisense oligomer” or “ASO,” as used herein, encompasses oligonucleotides and any other oligomeric molecule that comprises nucleobases capable of hybridizing to a complementary sequence on a target RNA transcript, including, but not limited to, those that do not comprise a sugar moiety, such as in the case of a peptide nucleic acid (PNA). Preferably, the ASO is an ASO that is resistant to nuclease cleavage or degradation.

The phrase “binds to a targeted portion” or “binds within a targeted portion,” in reference to an ASO or antisense RNA (AR), as used herein, refers to specific hybridization between the ASO or AR nucleotide sequence and a target nucleotide sequence that is complementary within the ranges set forth herein. In some examples, specific hybridization occurs where, under ex vivo conditions, the hybridization occurs under high stringency conditions. By “high stringency conditions” is meant that the ASO or AR, under such ex vivo conditions, hybridize to a target sequence in an amount that is detectably stronger than non-specific hybridization. High stringency conditions, then, are conditions that distinguish a polynucleotide with an exact complementary sequence, or one containing only a few scattered mismatches from a random sequence that happened to have a few small regions (e.g., 1-5 bases) that matched the probe. Such small regions of complementarity are more easily melted than a full-length complement of 12-17 or more bases, and moderate stringency hybridization makes them easily distinguishable. In one example, high stringency conditions include, for example, low salt and/or high temperature conditions, such as provided by about 0.02-0.1 M NaCl or the equivalent, at temperatures of about 50-70° C. The skilled person will appreciate that under in vivo conditions, the specificity of hybridization between an ASO or an AR and its target sequence is defined in terms of the level of complementarity between the ASO or an AR and the target sequence to which it hybridizes within a cell.

The term “peptide” is intended to include compounds composed of amino acid residues linked by amide bonds. A peptide may be natural or unnatural, ribosome encoded or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s). The peptide may comprise or consist of fewer than about 150 amino acids or fewer than about 125 amino acids or fewer than about 100 amino acids or fewer than about 90 amino acids or fewer than about 80 amino acids or fewer than about 70 amino acids or fewer than about 60 amino acids or fewer than about 50 amino acids.

Peptides, as referred to herein, include “inverso” peptides in which all L-amino acids are substituted with the corresponding D-amino acids, “retro-inverso” peptides in which the sequence of amino acids is reversed and all L-amino acids are replaced with D-amino acids.

Peptides may comprise amino acids in both L-and/or D-form. For example, both L-and D-forms may be used for different amino acids within the same peptide sequence. In some examples the amino acids within the peptide sequence are in L-form, such as natural amino acids. In some examples the amino acids within the peptide sequence are a combination of L-and D-form. Further, peptides may comprise unusual, but naturally occurring, amino acids including, but not limited to, hydroxyproline (Hyp), beta-alanine, citrulline (Cit), ornithine (Orn), norleucine (Nle), 3-nitrotyrosine, nitroarginine, pyroglutamic acid (Pyr). Peptides may also incorporate unnatural amino acids including, but not limited to, homo amino acids, N-methyl amino acids, alpha-methyl amino acids, beta (homo) amino acids, gamma amino acids, and N-substituted glycines. Peptides may be linear peptides or cyclic peptides.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical bond or a disulfide bond. Examples of non-covalent bonds include hydrogen bonds, ionic bonds, Van der Waals forces, and hydrophobic interactions.

Percentage amino acid sequence identity with respect to a given amino acid sequence is defined as the percentage of amino acid residues in a candidate sequence that are identical to the amino acid residues in the reference sequence, after aligning the sequences and introducing gaps, if necessary, to achieve the maximum percent sequence identity, and not considering any conservative substitutions as part of the sequence identity. Amino acid sequence identity may be determined using the EMBOSS Pairwise

Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5.

The default matrix “Blosum62” is utilized for amino acid sequences and the default matrix. Percent (%) or percentage “nucleic acid sequence identity” with respect to the nucleotide sequences disclosed herein is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent nucleic acid sequence identity can be achieved in various ways that are known in the art, for instance, using publicly available computer software such as BLAST or ALIGN. The skilled person can readily determine appropriate parameters for measuring alignment, including any algorithms needed to achieve maximal alignment over the full length of the sequences being compared.

The term “cell penetrating peptide” (CPP) refers to a peptide that is capable of crossing a cellular membrane. In one example, a CPP is capable of translocating across a mammalian cell membrane and entering into a cell. In another example, a CPP may direct a conjugate to a desired subcellular compartment. Thus, a CPP may direct or facilitate penetration of a molecule of interest across a phospholipid, mitochondrial, endosomal, lysosomal, vesicular, or nuclear membrane. A CPP may be translocated across the membrane with its amino acid sequence complete and intact, or alternatively partially degraded.

A CPP may direct a molecule of interest, such as an ASO disclosed herein, from outside a cell through the plasma membrane, and into the cytoplasm or a desired subcellular compartment. Alternatively, or in addition, a CPP may direct a molecule of interest across the blood-brain, trans-mucosal, hematoretinal, skin, gastrointestinal and/or pulmonary barriers.

The term “peptide ligand” or “receptor binding domain” refers to a peptide that is capable of binding to a membrane surface receptor to enable translocation of the peptide across a cellular membrane. In one example a peptide ligand may enable translocation across the cellular membrane via the natural endocytosis of the targeted receptor. In another example the peptide ligand may utilise a complementary mechanism of translocation across the cellular membrane including utilising a conjugated CPP. In one example, a peptide ligand is capable of translocating across a mammalian cell membrane and to enter a cell. In another example, a peptide ligand may direct a conjugate to a desired subcellular compartment. Thus, a peptide ligand may direct or facilitate cellular uptake of a molecule of interest across a phospholipid, mitochondrial, endosomal, lysosomal, vesicular, or nuclear membrane. A peptide ligand may be translocated across the membrane with its amino acid sequence complete and intact, or alternatively partially degraded.

A peptide ligand via its binding to a target receptor may direct a molecule of interest, such as an ASO disclosed herein, from outside a cell through the plasma membrane, and into the cytoplasm or a desired subcellular compartment. Alternatively, or in addition, a peptide ligand via its binding to a target receptor may direct a molecule of interest across a relevant biological barrier, e.g., the blood-brain, trans-mucosal, hematoretinal, skin, gastrointestinal, and/or pulmonary barriers.

### Compositions for Increasing OPA1 mRNA and or Protein Levels

ADOA is characterized by degeneration of retinal ganglion cells, and at least 75% of cases are caused by mutations in the OPA1 gene. OPA1 is ubiquitously expressed and is most abundant in the retina. The OPA1 protein is a mitochondrial GTPase that plays an important role in mitochondrial structure, function and control of apoptosis.

The unique distribution of mitochondria in retinal ganglion cells points to a critical function of this network in retinal ganglion cells and may underlie the sensitivity of these cells to OPA1 loss (also potentially precipitated by exposure of these cells to photo-oxidative stress). The majority of OPA1 mutations (˜27% missense, 27% splice variant, 23.5% frameshift, 16.5% nonsense, 6% deletion or duplication) lead to degradation of the transcript by mRNA decay, supporting the hypothesis that haploinsufficiency is a major mechanism underlying ADOA pathogenesis. OPA1 mutations leading to haploinsufficiency cause impaired mitochondrial function including defective complex I-driven ATP synthesis. As ATP is the principal energy currency in cells, insufficient ATP ultimately leads to cellular malfunction and death.

OPA1 haploinsufficiency accounts for a significant proportion of ADOA. OPA1 haploinsufficiency causing ADOA is amenable to treatment by upregulating wild type OPA1.

The invention provides a means to increase levels of functional OPA1 protein expression. Preferably the increased functional OPA1 is in patients who have ADOA, more preferably in ADOA where OPA 1 haploinsufficiency is implicated.

The term “nonsense-mediated RNA decay-inducing (NMD) exon” or “NMD exon” refers to an exon or a pseudo-exon that lies in a region within an intron and can activate the NMD pathway if included in a mature RNA transcript. The NMD pathway is triggered when the exon or pseudo exon either contains a premature termination codon (PTC) or induced a frameshift in the transcript. In the constitutive splicing events, the intron containing an NMD exon is usually spliced out, but the intron or part thereof can be retained during alternative or aberrant splicing events. Mature mRNA transcripts containing such an NMD exon can be non-productive due to a reading frame shift that induces the NMD pathway. Inclusion of a NMD exon in mature OPA1 RNA transcripts can downregulate overall OPA1 mRNA and protein expression.

Introns are removed by a large RNA-protein complex termed the spliceosome, which orchestrates complex interactions between primary transcripts, small nuclear RNAs (snRNAs) and a large number of proteins. Spliceosomes assemble on each intron in an ordered manner, starting with recognition of the 5′ splice site (5′ss) by U1 snRNA or the 3′ splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65) that binds the polypyrimidine tract (PPT), and a U2AF1-encoded 35-kD subunit (U2AF35) that interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes that have the same sequences but outnumber authentic sites by an order of magnitude.

Recently, it was recognized that, for many mammalian genes, mis-splicing gives rise to one or more NMD transcripts. Thus, it follows that in the case of such a gene, the steady state level of productive RNA transcripts and functional protein for that gene is reduced in proportion to the prevalence of NMD transcripts. Despite such “inefficiencies”, in a genetic background of two functional (wild type) alleles, the level of productive RNA transcripts and translation yields a sufficient level of functional protein for a given gene. However, in the case of certain, monogenic diseases, the loss of one functional allele results in haploinsufficiency and the associated disease. Thus, useful strategies for treatment of such diseases seek to increase the level of the relevant protein, expressed from the remaining healthy allele, in the affected target tissues. Recently, it has been shown that for a given gene of interest, a pre-mRNA can be targeted with antisense oligonucleotides that can reduce the level of mis-splicing of that pre-mRNA and increase the number of productive transcripts (Lim et al., 2020, Nature Communications, 11:3501).

The strategy of using an ASO to exclude a mutation induced retained intron with a PTC has been used to treat β-thalassemia (Seirakowska et al; Proc Natl Acad Sci USA 1996; 93(23): 12840-12844); cystic fibrosis (Friedman et al; Nucleic Acids, Protein Synthesis, and Molecular Genetics 1999; 274(51): 36193-36199) and ataxia-telangiectasia (Du et al; Proc Natl Acad Sci USA 2007 Apr. 3; 104(14): 6007-6012). Further use of PTC containing introns as a gene expression regulatory mechanism has been reviewed in Lewis et al (Proc Natl Acad Sci USA 2003; 100(1): 189-92), Mendell et al Science 2002; 298(5592): 419-22), Ni et al (Genes Dev 2007; 21(6): 708-718), Wong et al (Cell 2013; 154:583-595), and Aartsma-Rus and Ommen (RNA 2007; 13(10): 1609-1624).

The terms “antisense oligonucleotide”, “antisense oligonucleotide”, “oligonucleotide” and “antisense compound” may be used interchangeably to refer to an oligonucleotide.

While not wishing to be bound by theory, it is believed that an antisense sequence can sterically hinder access of the splicesome to a targeted splice site sequence by binding at or near the splicing motifs.

The OPA1 gene contains an intron with a premature termination codon (PTC) in intron 7 (located between exons 7 and 8). In all subjects, a proportion of the OPA1 RNA transcripts from wild-type OPA1 genes retain a section of intron 7 containing this PTC; this retained intron section is called exon 7x in the transcribed RNA. The RNA transcripts that contain exon 7x (the retained intron segment containing the PTC) are subject to nonsense-mediated RNA decay. Therefore, in all subjects there is a proportion of OPA1 RNA that is translated to mature wild-type protein, and a portion of OPA1 RNA that is degraded by RNase almost immediately due to the presence of the PTC. This immediately degraded OPA1 RNA does not affect the vision of a normal subject, as the combined functional protein production from the two available healthy alleles is sufficient for normal healthy function in the eye. Recently, exon 7 of OPA1 has also been referred to as exon 5b and intron 7x has been referred to as intron 5x. Thus, reference herein to exon 7 will also be a reference to exon 5 and reference to intron 7x will also be a reference to intron 5x.

However, if a subject has a mutation in one allele of the OPA1 gene, they are relying on the production of normal functional protein from their sole remaining non-mutated OPA1 gene. The amount of functional OPA1 protein produced by the one wild-type gene is insufficient for the needs of the eye.

The present invention seeks to address this insufficiency by ensuring that an increased amount of the OPA1 transcript produced by the wild-type OPA1 gene lacks exon 7x. This would mean that an increased amount of the RNA transcribed from the wild-type OPA1 gene is able to be translated into a functional protein, as additional RNA is “rescued” from conversion into an RNA form subject to nonsense-mediated RNA decay (NMD) by the presence of the PTC in exon 7x. This additional functional protein is then able to compensate for the inability of the mutant allele to produce functional OPA1 protein.

### Antisense Oligonucleotides (ASOs) and Antisense RNAs (Ars)

The present invention seeks to provide one or more antisense oligonucleotides (ASOs) that will reduce or prevent the inclusion of the portion of intron 7 that becomes NMD-prone exon 7x in the mRNA transcript.

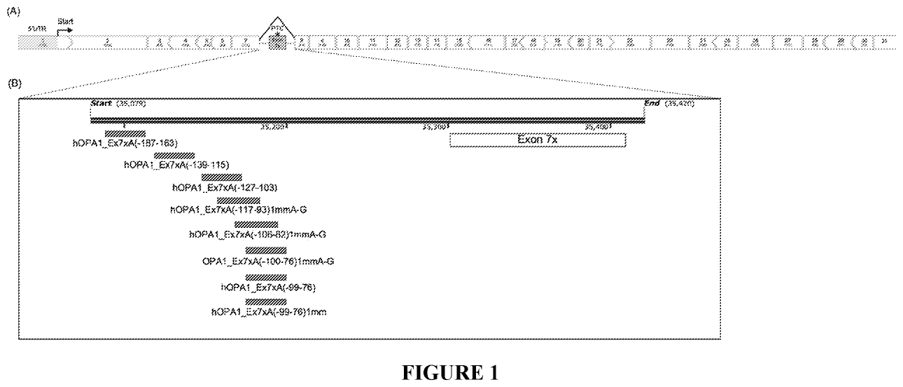

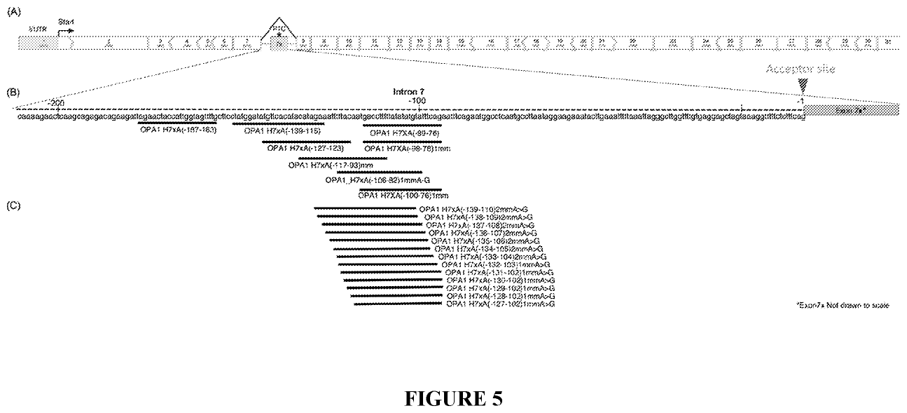

Antisense oligonucleotides were designed to bind to a target nucleic acid site in the OPA1 pre-mRNA transcript. Binding to a representative OPA1 transcript is shown in FIGS. 1 and 5, and pertains to all OPA1 transcripts.

Broadly, according to one aspect of the invention, there is provided an isolated or purified antisense oligonucleotide that targets intron 7 of the OPA1 gene transcript or part thereof. Preferably there is provided an isolated or purified antisense oligonucleotide to prevent aberrant retention of part or all of intron 7 during pre-mRNA splicing, thus avoiding production of OPA1 mRNA containing an NMD-prone, PTC containing aberrantly retained exon 7x that is derived from intron 7, and thereby resulting in increased translation of functional OPA1 protein. Preferably, there is provided an isolated or purified antisense oligonucleotide for inducing increased production of the OPA1 gene transcript or part thereof.

The antisense oligonucleotide may be selected from Table 1 and Table 2. Preferably, the antisense oligonucleotide is selected from the list comprising: SEQ ID Nos: 1-31; more preferably SEQ ID Nos: 1, 2, 3, 4 and 7; even more preferably SEQ ID Nos: 1, 3 and 4.

The antisense oligonucleotide may be selected from Tables 1, 2 and 4. Preferably, the antisense oligonucleotide is selected from the list comprising: SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

For example, in one aspect of the invention, there is provided an antisense oligonucleotide of 10 to 50 nucleotides comprising a targeting sequence complementary to a region near or within intron 7 of an OPA1 gene transcript or part thereof. In another aspect of the invention, there is provided an antisense oligonucleotide of 10 to 50 nucleotides comprising a targeting sequence complementary to a region near or within an exon of an OPA1 gene transcript or part thereof.

In one example, the nucleotide sequence of the ASO consists of 10 to 50 nucleotides, 15 to 40 nucleotides, 18 to 40 nucleotides, 17 to 25 nucleotides, 20 to 35 nucleotides, 20 to 30 nucleotides, 22 to 30 nucleotides, 22 to 28 nucleotides, 24 to 30 nucleotides, 25 to 30 nucleotides, or 26 to 30 nucleotides. In one example, the nucleotide sequence of the ASO consists of 20 to 30 nucleotides. For example, the nucleotide sequence of the ASO consists of 17 nucleotides. In one example, the nucleotide sequence of the ASO consists of 19 nucleotides. In another example, the nucleotide sequence of the ASO consists of 21 nucleotides. In a further example, the nucleotide sequence of the ASO consists of 22 nucleotides. In one example, the nucleotide sequence of the ASO consists of 23 nucleotides. In another example, the nucleotide sequence of the ASO consists of 24 nucleotides. In another example, the nucleotide sequence of the ASO consists of 25 nucleotides. In another example, the nucleotide sequence of the ASO consists of 26 nucleotides. In another example, the nucleotide sequence of the ASO consists of 27 nucleotides. In another example, the nucleotide sequence of the ASO consists of 28 nucleotides. In another example, the nucleotide sequence of the ASO consists of 29 nucleotides. In another example, the nucleotide sequence of the ASO consists of 30 nucleotides.

Broadly, according to one aspect of the invention, there is provided an isolated or purified antisense oligonucleotide designed to hybridise to a region within intron 7 of an OPA1 gene transcript or part thereof and prevent retention of all or part of intron 7. Antisense oligonucleotides (ASOs) were designed to target intron 7 with an aim to prevent retention of all or part of intron 7 including exon 7x by blocking intronic splice enhancers or other splicing motifs. This disruption will preferably modulate functional OPA1 protein expression by increasing the levels of correctly spliced wild-type OPA1 gene transcript and therefore levels of OPA1 protein. Increased OPA1 protein production may assist in treating a disease associated with OPA1 expression (particularly ADOA).

Broadly, according to another aspect of the invention, there is provided an isolated or purified antisense oligonucleotide designed to increase translation of functional OPA1 protein through binding to intron 7.

For example, in one aspect of the invention, there is provided an antisense oligonucleotide of 10 to 50 nucleotides comprising a targeting sequence complementary to a region near or within intron 7 of an OPA1 gene transcript or part thereof. In another aspect of the invention, there is provided an antisense oligonucleotide of 10 to 50 nucleotides comprising a targeting sequence complementary to a region near or within exon 7 of an OPA1 gene transcript or part thereof.

The antisense oligonucleotide may be selected from Table 1 and Table 2.Preferably, the antisense oligonucleotide is selected from the list comprising: SEQ ID Nos: 1-31; more preferably SEQ ID Nos: 1, 2, 3 and 4; even more preferably SEQ ID Nos: 1, 3 and 4.

The antisense oligonucleotide may be selected from Tables 1, 2 and 4. Preferably, the antisense oligonucleotide is selected from the list comprising: SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

Preferably the antisense oligonucleotide of the present invention operates to:
hybridise to intron 7 of the OPA1 gene transcript and prevent aberrant retention of part or all of intron 7 during pre-mRNA splicing, thus avoiding production of OPA1 mRNA containing an NMD-prone, PTC-containing aberrantly retained exon 7x that is derived from intron 7, and thereby resulting in increased translation of functional OPA1 protein

In the context of this document ‘aberrant splicing’ ‘aberrantly spliced’ and ‘aberrant retention’ refers to the alternative OPA1 gene transcript with retained all or part thereof intron 7. Intron retention may serve as a regulatory mechanism to fine-tune expression of various genes in certain cell types. Whether retention of all or part of OPA1 intron 7 serves this purpose is not known.

Preferably the antisense oligonucleotide of the present invention operates to:
restore mitochondrial structure
increase mitochondrial ATP levels; and/or
reduce apoptosis in the cells of patients with OPA1 mutations.

Preferably the antisense oligonucleotide of the present invention operates to:
restore mitochondrial structure
increase oxygen consumption rate
increase mitochondrial ATP levels; and/or
reduce apoptosis
   in the cells of patients with OPA1 mutations.

The antisense oligonucleotide of the present invention may include sequences that can hybridise to such sequences under stringent hybridisation conditions, sequences complementary thereto, sequences containing modified bases, modified backbones, and functional truncations or extensions thereof which possess or modulate splicing activity in a gene transcript. In certain embodiments, antisense oligonucleotides may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation that may occur in vivo. Hence, certain oligonucleotides may have about or at least about 70% sequence complementarity, e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

In one example, the nucleotide sequence of the ASO is at least 60%, 65%, 70%, 75%, 80%, 85%, 90%, 95%, or 100% complementary to the nucleotide sequence of the targeted portion over the length of the ASO.

The invention extends also to a combination of two or more antisense oligonucleotides capable of binding to a selected target to induce increased translation of functional OPA1 protein, including a construct comprising two or more such antisense oligonucleotides. The construct may be used for an antisense oligonucleotide-based therapy.

The invention extends, according to a still further aspect thereof, to cDNA or cloned copies of the antisense oligonucleotide sequences of the invention, as well as to vectors containing the antisense oligonucleotide sequences of the invention. The invention extends further also to cells containing such sequences and/or vectors.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of RNA, mRNA or protein, “isolating” refers to the recovery of RNA, mRNA or protein from a source, e.g., cells.

An antisense oligonucleotide can be said to be “directed to” or “targeted against” a target sequence with which it hybridizes. In certain embodiments, the target sequence includes a region near or within intron 7. The target sequence is preferably within intron 7, more preferably near the 5′ end of intron 7, and/or near the junction of exon 7 and intron 7. The antisense oligonucleotide may be selected from Table 1 and Table 2. Preferably, the antisense oligonucleotide is selected from the list comprising: SEQ ID Nos: 1-31; more preferably SEQ ID Nos: 1, 2, 3, 4 and 7; even more preferably SEQ ID Nos: 1, 3 and 4. The antisense oligonucleotide may be selected from Tables 1, 2 and 4. Preferably, the antisense oligonucleotide is selected from the list comprising: SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

In certain embodiments, the antisense oligonucleotide has sufficient sequence complementarity to a target RNA (i.e., the RNA for which translation is modulated) to block a region of a target RNA (e.g., mRNA) in an effective manner. In exemplary embodiments, such as binding to intron 7.

An antisense oligonucleotide having a sufficient sequence complementarity to a target RNA sequence to bind to parts of the target RNA means that the antisense oligonucleotide has a sequence sufficient to alter the three-dimensional structure of the targeted RNA.

Selected antisense oligonucleotides can be made shorter, e.g., about 12 bases, or longer, e.g., about 50 bases, and include a small number of mismatches, as long as the sequence is sufficiently complementary to effect mRNA structure upon hybridization to the target sequence, and optionally forms with the RNA a duplex having a Tm of 45° C. or greater.

Preferably, the antisense oligonucleotide is selected from the group comprising the sequences set forth in Table 1 and Table 2. Preferably, the antisense oligonucleotide is selected from the group comprising the sequences in SEQ ID Nos: 1-31; more preferably SEQ ID Nos: 1, 2, 3, 4 and 7; even more preferably SEQ ID Nos: 1, 3 and 4.

Preferably, the antisense oligonucleotide is selected from the group comprising the sequences set forth in Tables 1, 2 and 4. Preferably, the antisense oligonucleotide is selected from the group comprising the sequences in SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

In certain embodiments, the degree of complementarity between the target sequence and antisense oligonucleotide is sufficient to form a stable duplex. The region of complementarity of the antisense oligonucleotides with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An antisense oligonucleotide of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligonucleotide (PMO) antisense oligonucleotides, an optimum balance of binding stability and uptake generally occurs at lengths of 17-25 bases. Included are antisense oligonucleotides (e.g., CPP-PMOs, PPMOs, PMOs, PMO-X, PNAs, LNAs, 2′-Ome, 2′-MOE, 2′-F oligonucleotide, thiomorpholino and other hybrid oligonucleotide chemistries) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases. PMO—phosphorodiamidate morpholino oligonucleotide; CPP—cell penetrating peptide; PPMO—peptide-conjugated phosphorodiamidate morpholino oligonucleotide; PNA—peptide nucleic acid; LNA—locked nucleic acid; 2′-Ome-2′-O-methyl-modified oligonucleotide; 2′-MOE-2′-O-methoxy ethyl oligonucleotide, 2′-F-2′-Fluoro)

In certain embodiments, antisense oligonucleotides may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 60% sequence complementarity, e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C nucleobase pairs in the duplex, and the position of the mismatch(es) in the duplex, according to well understood principles of duplex stability. Although such an antisense oligonucleotide is not necessarily 100% complementary to the target sequence, it is effective to stably and specifically bind to the target sequence, such that the efficiency of mRNA translation is improved.

The stability of the duplex formed between an antisense oligonucleotide and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligonucleotides may have a binding Tm, with respect to a complementary-sequence RNA, of greater than body temperature and preferably greater than about 45° C. or 50° C. Tm's in the range 60-80° C. or greater are also included.

Additional examples of variants include antisense oligonucleotides having about or at least about 60% sequence identity, e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4.

In one example, antisense oligonucleotides of the disclosure have about or at least about 75% sequence identity, e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4. For example, antisense oligonucleotides of the disclosure have about or at least about 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4. In another example, antisense oligonucleotides of the disclosure have about or at least about 85% sequence identity, e.g., 85%, 86%, 87%, 88%, 89%, 90%, 91%, 92%, 93%. 94%, 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4. In a further example, antisense oligonucleotides of the disclosure have about or at least about 90% sequence identity, e.g., 90%, 91%, 92%, 93%, 94%, 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4. In one example, antisense oligonucleotides of the disclosure have about or at least about 95% sequence identity, e.g., 95%, 96%, 97%, 98%, 99% or 100% sequence identity, over the entire length of any of the sequences provided in Table 1 and Table 2 or any of SEQ ID Nos: 1-31; more preferably SEQ ID Nos: 1, 2, 3, 4 and 7; even more preferably SEQ ID Nos: 1, 3 and 4.

Additional examples of variants include antisense oligonucleotides having about or at least about 60% sequence identity, e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

In one example, antisense oligonucleotides of the disclosure have about or at least about 75% sequence identity, e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44. For example, antisense oligonucleotides of the disclosure have about or at least about 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44. In another example, antisense oligonucleotides of the disclosure have about or at least about 85% sequence identity, e.g., 85%, 86%, 87%, 88%, 89%, 90%, 91%, 92%, 93%, 94%, 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44. In a further example, antisense oligonucleotides of the disclosure have about or at least about 90% sequence identity, e.g., 90%, 91%, 92%, 93%, 94%, 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44. In one example, antisense oligonucleotides of the disclosure have about or at least about 95% sequence identity, e.g., 95%, 96%, 97%, 98%, 99% or 100% sequence identity, over the entire length of any of the sequences provided in Tables 1, 2 and 4 or any of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

More specifically, there is provided an antisense oligonucleotide capable of binding to a selected target site to increase exclusion of a nonsense-mediated RNA decay-inducing (NMD) exon in an OPA1 gene transcript or part thereof. The antisense oligonucleotide is preferably selected from those provided in Table 1 and Table 2 or SEQ ID Nos: 1-31; more preferably SEQ ID Nos: 1, 2, 3, 4 and 7; even more preferably SEQ ID Nos: 1, 3 and 4.

More specifically, there is provided an antisense oligonucleotide capable of binding to a selected target site to increase exclusion of a nonsense-mediated RNA decay-inducing (NMD) exon in an OPA1 gene transcript or part thereof. The antisense oligonucleotide is preferably selected from those provided in Tables 1, 2 and 4 or SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

The modification of mRNA translation preferably increases production of functional OPA1 protein through exclusion of a nonsense-mediated RNA decay-inducing (NMD) exon. The modification of mRNA translation of the present invention, using antisense oligonucleotides, may promote exclusion of nonsense-mediated RNA decay-inducing (NMD) exon 7x, derived from intron 7, during splicing of OPA1 pre-mRNA.

The antisense oligonucleotides of the invention may be a combination of two or more antisense oligonucleotides capable of binding to a selected target to induce exclusion of a nonsense-mediated RNA decay-inducing (NMD) exon. The combination may be a cocktail of two or more antisense oligonucleotides and/or a construct comprising two or more antisense oligonucleotides joined together.

In the present invention, antisense oligonucleotides are also known as antisense oligonucleotides, ASOs, Aos, AONs, antisense phosphorodiamidate morpholino oligomer, PMO, peptide-conjugated PMO and PPMO-the terms are interchangeable.

Preferably, the antisense oligonucleotide is selected from the group comprising the sequences set forth in Table 1 and Table 2. Preferably, the antisense oligonucleotide is selected from the group comprising the sequences in SEQ ID Nos: 1-31; more preferably SEQ ID Nos: 1, 2, 3, 4 and 7; even more preferably SEQ ID Nos: 1, 3 and 4.

Preferably, the antisense oligonucleotide is selected from the group comprising the sequences set forth in Tables 1, 2 and 4. Preferably, the antisense oligonucleotide is selected from the group comprising the sequences in SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

TABLE 1
List of antisense oligonucleotide sequences used in this study.

| SEQ | | | | |
|---|---|---|---|---|
| ID | Transcript Coordinates | Sequence 5′→3′ | Length | Efficacy |
| 1 | hOPA1_Ex7xA(-99-76) | CTGAAATACATATATAAAAAGGTC | 24 | +1 |
| 2 | hOPA1_Ex7xA(-99-76) | CTGAAATACATATATAAGAAGGTC | 24 | +1 |
| | 1 mm | | | |
| 3 | hOPA1_Ex7xA(−100-76) | CTGAAATACATATATAAGAAGGTC | 25 | +1 |
| | 1 mmA-G | A | | |
| 4 | hOPA1_Ex7xA(−106-82) | TACATATATAAGAAGGTCATTGTA | 25 | +1 |
| | 1 mmA-G | A | | |
| 5 | hOPA1_Ex7xA(−117-93) | AAAGGTCATTGTAAGAATTTCTAT | 25 | 0 |
| | 1 mmA-G | G | | |
| 6 | hOPA1_Ex7xA(−127-103) | GTAAAAATTTCTATGTATGTGAAC | 25 | 0 |
| | | A | | |
| 7 | hOPA1_Ex7xA(−139-115) | TCTATGTATGTGAACATATCCATA | 25 | +1 |
| | | G | | |
| 8 | hOPA1_Ex7xA(−187-163) | AATCTTCTGTCTCTGCTTGAGTTC | 25 | 0 |
| | | T | | |

The efficiency score of PMO-induced OPA1 upregulation; +1; greater than 1.1-fold OPA1 upregulation, 0; No OPA1 upregulation, −1; OPA1 downregulation.

TABLE 2
List of antisense oligonucleotide micro-walked
sequences with 17 nucleotides in length.

| SEQ | Transcript | | |
|---|---|---|---|
| ID | Coordinates | Sequence 5′→3′ | Length |
| 9 | OPA1 H7XA(-215-199) | CTCTGCTTGAGTTCTTT | 17 |
| 10 | OPA1 H7XA(-212-196) | TGTCTCTGCTTGAGTTC | 17 |
| 11 | OPA1 H7XA(-209-193) | TTCTGTCTCTGCTTGAG | 17 |
| 12 | OPA1 H7XA(-206-190) | ATCTTCTGTCTCTGCTT | 17 |
| 13 | OPA1 H7XA(-203-187) | CTAATCTTCTGTCTCTG | 17 |
| 14 | OPA1 H7XA(−164-148) | TGAACATATCCATAGGA | 17 |
| 15 | OPA1 H7XA(−161-145) | ATGTGAACATATCCATA | 17 |
| 16 | OPA1 H7XA(−158-142) | TGTATGTGAACATATCC | 17 |
| 17 | OPA1 H7XA(−155-139) | CTATGTATGTGAACATA | 17 |
| 18 | OPA1 H7XA(−152-136) | TTTCTATGTATGTGAAC | 17 |
| 19 | OPA1 H7XA(−149-133) | AAATTTCTATGTATGTG | 17 |
| 20 | OPA1 H7XA(−146-130) | TAAAAATTTCTATGTAT | 17 |
| 21 | OPA1 H7XA(−143-127) | TTGTAAAAATTTCTATG | 17 |
| 22 | OPA1 H7XA(−140-124) | TCATTGTAAAAATTTCT | 17 |
| 23 | OPA1 H7XA(−137-121) | AGGTCATTGTAAAAATT | 17 |
| 24 | OPA1 H7XA(−134-118) | AAAAGGTCATTGTAAAA | 17 |
| 25 | OPA1 H7XA(−131-115) | ATAAAAAGGTCATTGTA | 17 |
| 26 | OPA1 H7XA(−128-112) | TATATAAAAAGGTCATT | 17 |
| 27 | OPA1 H7XA(−125-109) | ACATATATAAAAAGGTC | 17 |
| 28 | OPA1 H7XA(−122-106) | AATACATATATAAAAAG | 17 |
| 29 | OPA1 H7XA(−119-103) | TGAAATACATATATAAA | 17 |
| 30 | OPA1 H7XA(−116-100) | TTCTGAAATACATATAT | 17 |
| 31 | OPA1 H7XA(−113-97) | AAATTCTGAAATACATA | 17 |

TABLE 3
OPA1 intron 7 (lowercase) and exon 7x (uppercase) cDNA sequence
(GRCh38/hg38: chr3 193626203-193628616)

| SEQ ID | |
|---|---|
| 32 | gtgatggatggtttaagggggctaccgatacattcacactaatcagccatttctgccaaga |
| | tcatgtcacctcaatctgttcatggactccaaatacaagaaattaatttgacaaagtgaaa |
| | atataaaagatgcatcatataaatatgtaacttttctggagtgggtagtataggtaaagcc |
| | aaaagaaacaaattcaagcagaggaattttggtttctgaaaattaggttgtctgtagggtc |
| | cctgtatttatacttagaacaaaattaggaatttctgtttatgtggtccagttattgagtc |
| | accctaagtttgtaggcatcttacctacctacttgctccccaagtttttatttctaaaatg |
| | aaaagcattgctgtagatgaccagtttacactaaagaataacatttatttatttgttttag |
| | ctaaagtatatggacagggaacattcatattcttgtagaagaaaattattttgacttttgg |
| | gcaaaagcatgtagttcttatacactttgacaaactcattgcgtacatttttcacattaat |
| | caaagtcagcacaaataaattttcaccttggaccacggagggtttgaacactggaaatttg |
| | atataattctggttgctaaagaacaagttctaataaaagcttaagtgtataccaatatgtg |
| | gctgttggtgcaatcagcaggtccgtaaaaatatgattttaatggttaggtaatcccacaa |
| | cggagatoccaaagttcatgtttggaagagacttttgggtcaaagtgaaatcagtgtaatg |
| | aatttaaaattatactctgagatcttgaaatcagctaattatgttacatcttattagctca |
| | gaaaagttttgaagttatatacaaatgctagtcaggaaaaaagattcagtcatgtaattct |
| | tgtacattctactatttaaatcaaccaatattatagattatgatttagtgcagtaattctg |
| | ctggctaaccttatctcatttggtggtggttagtacttcagagtactcaccatagtttcat |
| | ttatgttttcagcatcacttcctggtttttctcaattccatggctgtggaatcaattcata |
| | tgtatatttagcttcggtgagcaaaaacatagctagaaaaagaaaagaagtgagtttccta |
| | cctggttaaattaaagtcgatgtgttaagccaaggaggacttcttttgaatggtactttaa |
| | caatccctgttctgtatactgtgaatatatcatttaaatagcctaataaattggatgctta |
| | ggctgagccacctatactttagttttgttatggaaagaagggagaggagcaagtatgttct |
| | tatatgttacttagaaataagaatgtagctgtagttacacattgttcttaagtttttttcg |
| | taagacaacttgaaatgagtcccataggcctgctatttaacattctaagatatgacttaag |
| | gttaatgatgagcttttgaatctgacaattcaagagatatccataatgaatactgattcat |
| | tttctacattgctgaaagctaatgttcattttaagcctactttagtagcctttatttgggc |
| | ttagagatgttattcctctttctgatatttattgggttatctgtttaacccttttatatct |
| | ccctttcccgatttgtaaattagagactggcaagactttttaccctgagtagagcaccaaa |
| | catggcttgtttctgcccacactgtagttaccttgaggggaagtaaatgggactttaaaag |
| | caatttatgctcttttatagtgaaattatccctcttactatcccgaaagactgttacctta |
| | caatatcctccactcctttccccctgtagttactatagagatgacttttcggttcttcact |
| | gccataatgatcaaaatcctaattcatgagatttttatcattccaggcatgtgaggtttac |
| | ttgatgcataaaaccgcaagtactttttgttgttttttaattgttttttctctcttatctt |
| | cttgaaagtctaagtagatcatcatttttgatgtcttattagtagcaactaataaattttc |
| | cctgtatcttctcagcaaaagaactcaagcagagacagaagattagaactaccattggtag |
| | ttttgcttcctatggatatgttcacatacatagaaatttttacaatgacctttttatatat |
| | gtatttcagaatttcagaatggcctcaatgccttaataggaagaaatacttgaaattttta |
| | aattagggcttggttttgtgaggagctagtaaaggtttttctctttcagCTTTAGCTTGTT |
| | TCTGCGGAGGATTCCGCTCTTTCTCCATCAGTTTCATAGCCCTGGAATTGTAGAAAAGCTC |
| | TGGTTTCAAGACCATTGATATCCATTTCTGTCAGG |

TABLE 4
Refinement of antisense oligonucleotide sequences
targeting OPA1 intron 7.

| SEQ ID | Transcript Coordinates | Sequence 5′→3′ | Length |
|---|---|---|---|
| 33 | OPA1_Ex7xA(−127- | CTGAAATACATATATAAAAAGGTCA | 25 |
| | 102)1 mmA>G | | |
| 34 | OPA1_Ex7xA(−128- | CTGAAATACATATATAAAAAGGTCAT | 26 |
| | 102)1 mmA>G | | |
| 35 | OPA1_Ex7xA(−129- | CTGAAATACATATATAAAAAGGTCATT | 27 |
| | 102)1 mmA>G | | |
| 36 | OPA1_Ex7xA(−130- | CTGAAATACATATATAAAAAGGTCATTG | 28 |
| | 102)1 mmA>G | | |
| 37 | OPA1_Ex7xA(−131- | CTGAAATACATATATAAAAAGGTCATTGT | 29 |
| | 102)1 mmA>G | | |
| 38 | OPA1_Ex7xA(−132- | CTGAAATACATATATAAAAAGGTCATTGTA | 30 |
| | 103)1 mmA>G | | |
| 39 | OPA1_Ex7xA(−133- | TGAAATACATATATAAAAAGGTCATTGTAA | 30 |
| | 104)1 mmA>G | | |
| 40 | OPA1_Ex7xA(−134- | GAAATACATATATAAAAAGGTCATTGTAAA | 30 |
| | 105)2mmA>G | | |
| 41 | OPA1_Ex7xA(−135- | AAATACATATATAAAAAGGTCATTGTAAAA | 30 |
| | 106)2mmA>G | | |
| 42 | OPA1_Ex7xA(−136- | AATACATATATAAAAAGGTCATTGTAAAAA | 30 |
| | 107)2mmA>G | | |
| 43 | OPA1_Ex7xA(−137- | ATACATATATAAAAAGGTCATTGTAAAAAT | 30 |
| | 108)2mmA>G | | |
| 44 | OPA1_Ex7xA(−138- | TACATATATAAAAAGGTCATTGTAAAAATT | 30 |
| | 109)2mmA>G | | |
| 45 | OPA1_Ex7xA(−139- | ACATATATAAAAAGGTCATTGTAAAAATTT | 30 |
| | 110)2 mmA>G | | |
| 46 | OPA1_H7xA(−126-102) | CTGAAATACATATATAAAAAGGTCA | 25 |
| 47 | OPA1_H7xA(−132-108) | TACATATATAAAAAGGTCATTGTAA | 25 |
| 48 | OPA1_H7xA(−131-102) | CTGAAATACATATATAAAAAGGTCATTGTA | 30 |
| 49 | OPA1_H7xA(−132-103) | TGAAATACATATATAAAAAGGTCATTGTAA | 30 |
| 50 | OPA1_H7xA(−133-104) | GAAATACATATATAAAAAGGTCATTGTAAA | 30 |
| 51 | OPA1_H7xA(−134-105) | AAATACATATATAAAAAGGTCATTGTAAAA | 30 |
| 52 | OPA1_H7xA(−125-96) | GAAATTCTGAAATACATATATAAAAAGGTC | 30 |
| 53 | OPA1_H7xA(−126-97) | AAATTCTGAAATACATATATAAAAAGGTCA | 30 |
| 54 | OPA1_H7xA(−127-98) | AATTCTGAAATACATATATAAAAAGGTCAT | 30 |

SEQ ID No: 55 Exemplary of amino acid sequence of the CPP for PMO conjugation:

RRSRTARAGRPGRNSSRPSAPRGASGGASG

There is also provided a method for exclusion of an NMD exon in an OPA1 gene transcript, the method including the step of a) providing one or more of the antisense oligonucleotides as described herein and allowing the oligonucleotide(s) to bind to a target nucleic acid site.

According to yet another aspect of the invention, there is provided a target nucleic acid sequence for exclusion of an NMD exon in OPA1 comprising the DNA equivalents of the nucleic acid sequences selected from Table 1 and Table 2 or the group consisting of SEQ ID Nos: 1-31; more preferably SEQ ID Nos: 1, 2, 3, 4 and 7; even more preferably SEQ ID Nos: 1, 3 and 4.

According to yet another aspect of the invention, there is provided a target nucleic acid sequence for exclusion of an NMD exon in OPA1 comprising the DNA equivalents of the nucleic acid sequences selected from Tables 1, 2 and 4 or the group consisting of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

Designing antisense oligonucleotides to sterically hinder access of the splicesome to a targeted splice site sequence by it and/or in proximity to it may not necessarily generate a change in splicing of the targeted gene transcript. Furthermore, the inventors have discovered that size or length of the antisense oligonucleotide itself is not always a primary consideration when designing antisense oligonucleotides. With some targets, antisense oligonucleotides as short as 17 bases were able to induce some change in splicing, in certain cases more efficiently than other longer (e.g. 25 bases) oligonucleotides directed to the same regulatory element.

The inventors have also discovered that there does not appear to be any standard motif that can be blocked or masked by antisense oligonucleotides to redirect splicing. Although tool-assisted analysis of RNA can predict likely functional motifs in the RNA sequence, it was found that antisense oligonucleotides must be designed, and their individual efficacy evaluated empirically.

More specifically, the antisense oligonucleotide may be selected from those set forth in Table 1 and Table 2. The sequences are preferably selected from the group consisting of any one or more of SEQ ID Nos: 1-31; more preferably SEQ ID Nos: 1, 2, 3, 4 and 7; even more preferably SEQ ID Nos: 1, 3 and 4, and combinations or cocktails thereof. More specifically, the antisense oligonucleotide may be selected from those set forth in Tables 1, 2 and 4. The sequences are preferably selected from the group consisting of any one or more of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44, and combinations or cocktails thereof. This includes sequences that can hybridise to such sequences under stringent hybridisation conditions, sequences complementary thereto, sequences containing modified bases, modified backbones, and functional truncations or extensions thereof which possess or modulate retained intron retention activity in an OPA1 gene transcript.

The oligonucleotide and the DNA, cDNA or RNA are complementary to each other when a sufficient number of corresponding positions in each molecule are occupied by nucleotides which can hydrogen bond with each other. Thus, “specifically hybridisable” and “complementary” are terms used to indicate a sufficient degree of complementarity or pairing such that stable and specific binding occurs between the oligonucleotide and the DNA, cDNA or RNA target. It is understood in the art that the sequence of an antisense oligonucleotide need not be 100% complementary to that of its target sequence to be specifically hybridisable. In certain examples, the nucleotide sequences of ASOs in the compositions disclosed herein can be at least 60%, at least 65%, at least 70%, at least 75%, at least 80%, at least 85%, at least 90%, at least 95%, or 100% complementary to the nucleotide sequence of the targeted portion of an RNA transcript over the length of the ASO nucleotide sequence. For example, an ASO in which 18 of 20 nucleotides of ASO sequence are complementary to a target region, and would therefore specifically hybridize, would represent 90 percent complementarity. In such an example, the remaining non-complementary nucleotides of the ASO could be clustered together or interspersed with complementary nucleotides and need not be contiguous. Complementarity of an ASO sequence to a target nucleotide sequence (expressed as “percent complementarity” to its target sequence; or “percent identity” to its reverse complement sequence) can be determined routinely using algorithms known in the art, as exemplified in the BLAST programs (basic local alignment search tools) and PowerBLAST programs (Altschul, et al., 1990, J. Mol. Biol., 215:403-410; Zhang et al., 1997, Genome Res., 7:649-656). An antisense oligonucleotide is specifically hybridisable when binding of the compound to the target DNA or RNA molecule interferes with the function of the target DNA or RNA product, and there is a sufficient degree of complementarity to avoid non-specific binding of the antisense oligonucleotide to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the antisense oligonucleotides of the present invention may include oligonucleotides that selectively hybridise to the sequences provided in Table 1 and Table 2, or SEQ ID Nos: 1-31; more preferably SEQ ID Nos: 1, 2, 3, 4 and 7; even more preferably SEQ ID Nos: 1, 3 and 4. The antisense oligonucleotides of the present invention may also include oligonucleotides that selectively hybridise to the sequences provided in Tables 1, 2 and 4, or SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligonucleotide. The length of identity comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the antisense oligonucleotide sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, identity. Generally, the shorter the length of the antisense oligonucleotide, the greater the identity required to obtain selective hybridisation. Consequently, where an antisense oligonucleotide of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligonucleotides set out in the sequence listings herein. Nucleotide identity comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The antisense oligonucleotide of the present invention may have regions of reduced identity, and regions of exact identity with the target sequence. It is not necessary for an oligonucleotide to have exact identity for its entire length. For example, the oligonucleotide may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligonucleotide may have stretches of at least 10, 11, 12, 13, 14, 15, 16, 17, 18, 19, 20, 21, 22, 23, 24, 25 or 26 bases that are identical to the target sequence. The remaining stretches of oligonucleotide sequence may be intermittently identical with the target sequence: for example, the remaining sequence may have an identical base, followed by a non-identical base, followed by an identical base. Alternatively (or as well) the oligonucleotide sequence may have several stretches of identical sequence (for example 3, 4, 5 or 6 bases) interspersed with stretches of less than perfect identity. Such sequence mismatches will preferably have no or very little loss of splice modulating activity. Even more preferably, such sequence mismatches will lead to increased activity to reduce intron retention.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating”, or “augment” or “augmenting” refer generally to the ability of one or more antisense oligonucleotides or compositions to produce or cause a greater physiological response (i.e., downstream effects) in a cell or a subject relative to the response caused by either no antisense oligonucleotide or a control compound. The terms “decreasing” or “decrease” refer generally to the ability of one or antisense oligonucleotides or compositions to produce or cause a reduced physiological response (i.e., downstream effects) in a cell or a subject relative to the response caused by either no antisense oligonucleotide or a control compound.

Relevant physiological or cellular responses (in vivo or in vitro) will be apparent to persons skilled in the art and may include increases in the expression of functional OPA1 protein in a cell, tissue, or subject in need thereof. An “increased” or “augmented” amount may be a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) more than the amount produced when no antisense oligonucleotide is present (the absence of an agent) or a control compound is used.

The term “reduce” or “inhibit” may relate generally to the ability of one or more antisense oligonucleotides or compositions to “decrease” a relevant physiological or cellular response, such as a symptom of a disease described herein, as measured according to routine techniques in the diagnostic art. Relevant physiological or cellular responses (in vivo or in vitro) will be apparent to persons skilled in the art, and may include reductions in the symptoms or pathology of a disease such as ADOA.

A “decrease” in a response may be statistically significant as compared to the response produced by no antisense oligonucleotide or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligonucleotide may vary, as long as it is capable of binding selectively to the intended location within the mRNA molecule. The length of such sequences can be determined in accordance with selection procedures described herein. Generally, the antisense oligonucleotide will be from about 10 nucleotides in length, up to about 50 nucleotides in length. It will be appreciated, however, that any length of nucleotides within this range may be used in the method. Preferably, the length of the antisense oligonucleotide is between 10 and 40, 10 and 35, 15 to 30 nucleotides in length or 20 to 30 nucleotides in length, most preferably about 25 to 30 nucleotides in length. For example, the oligonucleotide may be 20, 21, 22, 23, 24, 25, 26, 27, 28, 29 or 30 nucleotides in length.

In some examples, the ASO nucleotide sequence is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 20 to 50 nucleotides, 20 to 40 nucleotides, 20 to 35 nucleotides, 20 to 30 nucleotides, 20 to 25 nucleotides, 25 to 50 nucleotides, 25 to 40 nucleotides, 25 to 35 nucleotides, or 25 to 30 nucleotides in length. In some examples, the ASOs are 17 nucleotides in length. In some preferred examples, the nucleotide sequence of the ASO nucleotide is 25 nucleotides in length.

As used herein, an “antisense oligonucleotide” refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The cyclic subunits may be based on ribose or another pentose sugar or, in certain embodiments, a morpholino group (see description of morpholino oligonucleotides below). Also contemplated are peptide nucleic acids (PNAs) or other artificial entity that allows a polynucleotide-like structure, locked nucleic acids (LNAs), 2′-O-methyl oligonucleotides, other antisense agents known in the art.

Included are non-naturally occurring antisense oligonucleotides, or “oligonucleotide analogs”, including antisense oligonucleotides or oligonucleotides having (i) a modified backbone structure, e.g., a backbone other than the standard phosphodiester linkage found in naturally occurring oligo-and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing antisense oligonucleotides is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA and/or mRNA during duplex formation with the antisense oligonucleotides, the antisense oligonucleotides used in the method may be adapted to minimise or prevent cleavage by endogenous RNase H. This property is highly preferred, as the treatment of the RNA with the unmethylated oligonucleotides, either intracellular or in crude extracts that contain RNase H, leads to degradation of the pre-mRNA:antisense and/or mRNA:antisense oligonucleotide duplexes. Any form of modified antisense oligonucleotides that is capable of by-passing or not inducing such degradation may be used in the present method. The nuclease resistance may be achieved by modifying the antisense oligonucleotides of the invention so that it comprises partially unsaturated aliphatic hydrocarbon chain and one or more polar or charged groups including carboxylic acid groups, ester groups, and alcohol groups.

Antisense oligonucleotides that do not activate RNase H can be made in accordance with known techniques (see, e.g., U.S. Pat. 5,149,797). Such antisense oligonucleotides, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense oligonucleotides that do not activate RNase H are available. For example, such antisense oligonucleotides may be oligonucleotides wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such antisense oligonucleotides are molecules wherein at least one, or all, of the nucleotides contain a 2′ lower alkyl moiety (such as, for example, C1-C4, linear or branched, saturated or unsaturated alkyl, such as methyl, ethyl, ethenyl, propyl, 1-propenyl, 2-propenyl, and isopropyl). For example, every other one of the nucleotides may be modified as described.

An example of antisense oligonucleotides, which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo-or deoxyribo-counterparts. Alternatively, the nuclease resistant antisense oligonucleotides of the invention may have at least one of the last 3′-terminus nucleotides fluoridated. Still alternatively, the nuclease resistant antisense oligonucleotides of the invention have phosphorothioate bonds linking between at least two of the last 3′-terminus nucleotide bases, preferably having phosphorothioate bonds linking between the last four 3′-terminal nucleotide bases.

Increased modulation of translation may also be achieved with alternative oligonucleotide chemistries. For example, the antisense oligonucleotide may be chosen from the list comprising: phosphoramidate or phosphorodiamidate morpholino oligonucleotide (PMO); PMO-X; PPMO; peptide nucleic acid (PNA); a locked nucleic acid (LNA) and derivatives including alpha-L-LNA, 2′-amino LNA, 4′-methyl LNA and 4′ O-methyl LNA; ethylene bridged nucleic acids (ENA) and their derivatives; phosphorothioate oligonucleotide; tricyclo-DNA oligonucleotide (tcDNA); tricyclophosphorothioate oligonucleotide; 2′-O-methyl-modified oligonucleotide (2′-O-Me); 2′-O-methoxy ethyl (2′-MOE); 2′-fluoro (2′-F), 2′-fluroarabino (FANA); unlocked nucleic acid (UNA); hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above-mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping antisense oligonucleotides. Antisense oligonucleotide-induced translation modulation of the human OPA1 gene transcripts have generally used either oligoribonucleotides, PNAs, 2′-O-Me or MOE modified bases on a phosphorothioate backbone. Although 2′-O-Me ASOs are used for oligo design, due to their efficient uptake in vitro when delivered as cationic lipoplexes, these compounds are susceptible to nuclease degradation and are not considered ideal for in vivo or clinical applications. When alternative chemistries are used to generate the antisense oligonucleotides of the present invention, the thymine (T) of the sequences provided herein may be replaced by a uracil (U).

While the antisense oligonucleotides described above are a preferred form of the antisense oligonucleotides of the present invention, the present invention includes other oligonucleotideic antisense molecules, including but not limited to oligonucleotide mimetics such as are described below.

Specific examples of preferred antisense oligonucleotides useful in this invention include oligonucleotides containing modified backbones or non-natural inter-nucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their inter-nucleoside backbone can also be considered to be antisense oligonucleotides.

In other preferred oligonucleotide mimetics, both the sugar and the inter-nucleoside linkage, i.e., the backbone, of the nucleotide units are replaced with novel groups. The base units are maintained for hybridization with an appropriate nucleic acid target compound. One such oligonucleotideic compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligonucleotide (PMO) compounds that are not degraded by any known nuclease or protease. These compounds are uncharged, and do not activate RNase H activity when bound to an RNA strand.

Modified oligonucleotides may also contain one or more substituted sugar moieties. Oligonucleotides may also include nucleobase (often referred to in the art simply as “base”) modifications or substitutions. Certain nucleobases are particularly useful for increasing the binding affinity of the oligonucleotide compounds of the invention. These include 5-substituted pyrimidines, 6-azapyrimidines, and N-2, N-6 and O-6 substituted purines, including 2-aminopropyladenine, 5-propynyluracil and 5-propynylcytosine, 5-methylcytosine substitutions have been shown to increase nucleic acid duplex stability by 0.6-1.2° C., even more particularly when combined with 2′-O-methoxyethyl sugar modifications.

Another modification of the oligonucleotides of the invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethy lammonium 1,2-di-O-hexadecyl-rac-glycero-3-H-phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligonucleotides to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligonucleotide. The present invention also includes antisense oligonucleotides that are chimeric compounds. “Chimeric” antisense oligonucleotides or “chimeras,” in the context of this invention, are antisense oligonucleotides, particularly oligonucleotides that contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide or antisense oligonucleotide increased resistance to nuclease degradation, increased cellular uptake, and an additional region for increased binding affinity for the target nucleic acid.

The activity of antisense oligonucleotides and variants thereof can be assayed according to routine techniques in the art. The expression levels of surveyed RNAs and proteins may be assessed by any of a wide variety of well-known methods for detecting expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays. Protein expression levels can be assessed by western blot and/or ELISA assays from a cell, tissue or organism, and by assessing downstream functional or physiological effects.

If two or more different sized transcripts are present, the resulting proteins may be assessed by any of a wide variety of well-known methods for detecting the expression of the relevant protein. Non-limiting examples of such methods include immunological methods for detection of proteins; protein purification methods; mass spectrometry; and protein function or activity assays.

The present invention provides antisense oligonucleotide induced pre-mRNA sequence modulation of the OPA1 gene transcript, clinically relevant oligonucleotide chemistries and delivery systems to direct exon structure, and functional OPA1 protein to therapeutic levels. Clinically relevant increases in the amount of functional OPA1 translation, and hence OPA1 protein from OPA1 gene transcription, are achieved by:
1) oligonucleotide refinement in vitro using fibroblast cell lines, through experimental assessment of (i) target motifs, (ii) antisense oligonucleotide length and development of oligonucleotide cocktails, (iii) choice of chemistry, and (iv) the addition of cell-penetrating peptides (CPP) to enhance oligonucleotide delivery; and
2) detailed evaluation of a novel approach to modulate OPA1 splicing via interaction with the OPA1 pre-mRNA transcript to exclude exon7X from the OPA mRNA transcripts

As such, it is demonstrated herein that splicing of OPA1 exon 7X can be modulated with specific antisense oligonucleotides. In this way functionally significant increases in the amount of OPA1 wild type mRNA transcript and OPA1 protein can be obtained, thereby reducing the severe pathology associated with ADOA caused by OPA1 haploinsufficiency.

The antisense oligonucleotides used in accordance with this invention may be conveniently made through the well-known technique of solid phase synthesis. Equipment for such synthesis is sold by several vendors including, for example, Applied Biosystems (Foster City, Calif.). One method for synthesising oligonucleotides on a modified solid support is described in U.S. Pat. No. 4,458,066.

Any other means for such synthesis known in the art may additionally or alternatively be employed. It is well known to use similar techniques to prepare oligonucleotides such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligonucleotides of the invention are synthesised in vitro and do not include antisense compositions of biological origin, or genetic vector constructs designed to direct the in vivo synthesis of antisense oligonucleotides. The molecules of the invention may also be mixed, encapsulated, conjugated or otherwise associated with other molecules, molecule structures or mixtures of compounds, as for example, liposomes, receptor targeted molecules etc.

The antisense oligonucleotides may be formulated for oral, topical, parenteral or other administration, particularly formulations for topical ocular and injectable ocular administration. The formulations may be formulated for assisting in uptake, distribution and/or absorption at the site of administration or activity. Preferably the antisense oligonucleotides of the present invention are formulated for delivered topically to the eye or by intraocular injection or intraocular implant, so that the effects on OPA1 production are spatially limited and are not systemic.

### ASO Chemistry and Modifications

The ASOs used in the compositions described herein may comprise naturally-occurring nucleotides, nucleotide analogs, modified nucleotides, or any combination thereof. The term “naturally occurring nucleotides” includes deoxyribonucleotides and ribonucleotides. The term “modified nucleotides” includes nucleotides with modified or substituted sugar groups and/or having a modified backbone. In some examples, all the nucleotides of an ASO are modified nucleotides. Chemical modifications of ASOs or components of ASOs that are compatible with the compositions and methods described herein are known in the art as disclosed in, e.g., in U.S. Pat. Nos. 8,258,109, 5,656,612, U.S. Patent Publication No. 2012/0190728, and Roberts et al., 2020, Nature Rev. Drug Disc., 19:673-694.

One or more nucleotides of an ASO may be any naturally occurring, unmodified nucleobase such as adenine, guanine, cytosine, thymine, uracil and inosine, or any synthetic or modified nucleobase that is sufficiently similar to an unmodified nucleobase such that it is capable of hydrogen bonding with a nucleobase present on a target RNA transcript. Examples of suitable modified nucleobases include, but are not limited to, hypoxanthine, xanthine, 7-methylguanine, 5, 6-dihydrouracil, 5-methylcytosine, and 5-hydroxymethoylcytosine.

ASOs include a “backbone” structure, that refers to the connection between nucleotides/monomers of the ASO. In naturally occurring oligonucleotides, the backbone comprises a 3′-5′ phosphodiester linkage connecting sugar moieties of adjacent nucleotides. Suitable types of backbone linkages for the ASOs described herein include, but are not limited to, phosphodiester, phosphorothioate, phosphorodithioate, phosphorodiamidate, phosphoroselenoate, phosphorodiselenoate, phosphoroanilothioate, phosphoraniladate, phosphoramidate, and the like. In some examples, the backbone modification is a phosphorothioate linkage. In other examples, the backbone modification is a phosphorodiamidate linkage. See, e.g., Roberts et al. supra; and Agrawal (2021), Biomedicines, 9:503. In some examples, the backbone structure of the ASO does not contain phosphorous-based linkages, but rather contains peptide bonds, for example in a peptide nucleic acid (PNA), or linking groups including carbamate, amides, and linear and cyclic hydrocarbon groups.

In some examples, the stereochemistry at each of the phosphorus internucleotide linkages of the ASO backbone is random. In other examples, the stereochemistry at each of the phosphorus internucleotide linkages of the ASO backbone is controlled and is not random. For example, U.S. Pat. No. 9,605,019 describes methods for independently selecting the handedness of chirality at each phosphorous atom in an oligonucleotide. In some examples, an ASO used in the compositions and methods provided herein, including, but not limited to, the ASOs the sequences of which are disclosed herein as SEQ ID Nos: 1-31 and 33-54 is an ASO having phosphodiester internucleotide linkages that are not random. In some examples, a composition or composition used in the methods disclosed herein comprises a pure diastereomeric ASO. In other examples, the composition comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some examples, the ASO has a non-random mixture of Rp and Sp configurations at its phosphorus internucleotide linkages. In some examples, an ASO used in the compositions and methods disclosed herein, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp.

In some examples, the ASOs described herein contain a sugar moiety that comprises ribose or deoxyribose, or a modified sugar moiety or sugar analog, including a morpholine ring. Suitable examples of modified sugar moieties include, but are not limited to 2′ substitutions such as 2′-O-modifications, 2′-O-methyl (2′-O-Me), 2′-O-methoxyethyl (2′MOE), 2′-O-aminoethyl, 2′F, N3′-P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some examples, the sugar moiety modification is selected from among 2′-O-Me, 2′F, and 2′MOE. In other examples, the sugar moiety modification is an extra bridge bond, such as in a locked nucleic acid (LNA). In some examples the sugar analogue contains a morpholine ring, such as phosphorodiamidate morpholino (PMO). In some examples, the sugar moiety comprises a ribofuransyl or 2′deoxyribofuransyl modification. In some examples, the sugar moiety comprises 2′4′-constraine, 2′-O-methyloxyethyl (cMOE) modifications. In some examples, the sugar moiety comprises cE′ 2′ 4′ constraine, 2′-O ethyl BNA modifications. In other examples, the sugar moiety comprises tricycloDNA (tcDNA) modifications. In some examples, the sugar moiety comprises ethylene nucleic acid (ENA) modifications. In some examples, the sugar moiety comprises 2′-O-(2-N-methylcarbamoylethyl) (MCE). Modifications are known in the art as exemplified in Jarver, et al., 2014, Nucleic Acid Therapeutics, 24(1): 37-47.

In some examples, each constituent nucleotide of the ASO is modified in the same way, e.g., every linkage of the backbone of the ASO comprises a phosphorothioate linkage, or each ribose sugar moiety comprises a 2′-O-methyl modification. In other examples, a combination of different modifications is used, e.g., an ASO comprising a combination of phosphorodiamidate linkages and sugar moieties comprising morpholine rings (morpholinos).

In some examples, the ASO comprises one or more backbone modifications. In some examples, the ASO comprises one or more sugar moiety modification. In some examples, the ASO comprises one or more backbone modifications and one or more sugar moiety modifications. In some examples, the ASO comprises a 2′MOE modification and a phosphorothioate backbone. In some examples, the ASO comprises a peptide nucleic acid (PNA).

In some preferred examples, the ASO comprises a phosphorodiamidate morpholino (PMO).

The skilled person in the art will appreciate that ASOs may be modified in order to achieve desired properties or activities of the ASO or reduce undesired properties or activities of the ASO. In some examples, an ASO is modified to alter one or more properties. For example, such modifications can: enhance binding affinity to a target sequence on a pre-mRNA transcript; reduce binding to any non-target sequence; reduce degradation by cellular nucleases (e.g., RNase H); improve uptake of an ASO into a cell and/or particular subcellular compartments; alter the pharmacokinetics or pharmacodynamics of the ASO; and/or modulate the half-life of the ASO in vivo.

In some examples, the ASOs comprise one or more 2′-O-(2-methoxyethyl) (MOE) phosphorothioate-modified nucleotides, which have been shown to confer significantly enhanced resistance of ASOs to nuclease degradation and increased bioavailability.

Methods for synthesis and chemical modification of ASOs, as well as synthesis of ASO conjugates is well known in the art, and such ASOs are available commercially.

In some examples, a composition (e.g., a pharmaceutical composition) provided here includes two or more ASOs with different chemistries but complementary to the same targeted portion of the OPA1 pre-mRNA. In other examples, two or more ASOs that are complementary to different targeted portions of the OPA1 pre-mRNA.

In some examples, the compositions disclosed herein include ASOs that are linked to a functional moiety. In some examples, the functional moiety is a delivery moiety, a targeting moiety, a detection moiety, a stabilizing moiety, or a therapeutic moiety. In some examples the functional moiety includes a delivery moiety or a targeting moiety. In some examples the functional moiety includes a stabilizing moiety. In some preferred examples the functional moiety is a delivery moiety.

Suitable delivery moieties include, but are not limited to, lipids, polyethers, peptides, carbohydrates, glycans, receptor binding domains (RBDs), and antibodies.

In some examples, the delivery moiety includes a cell-penetrating peptide (CPP). Suitable examples of CPPs are described in, e.g., PCT/AU2020/051397. In some examples the amino acid sequence of the CPP comprises or consists of: RRSRTARAGRPGRNSSRPSAPRGASGGASG (SEQ ID No: 55). In one example, the CPP comprises the sequence RRSRTARAGRPGRNSSRPSAPRGASGGASG (SEQ ID No: 55), optionally wherein any amino acid other than glycine is a D amino acid. In other examples, the delivery moiety includes a RBD.

In other examples, the delivery moiety includes a carbohydrate. In some examples, a carbohydrate delivery moiety is selected from among N-acetylgalactosamine (GalNAc), N-Ac-Glucosamine (GluNAc), and a mannose. In one example, the carbohydrate delivery moiety is GalNac.

In other examples, the delivery moiety includes a lipid. Examples of suitable lipids as delivery moieties include, but are not limited to, cholesterol moiety, a cholesteryl moiety, and aliphatic lipids. In some examples the delivery moiety includes a fatty acid or lipid moiety. In some embodiments the fatty acid chain length is about C8 to C20. Examples of suitable fatty acid moieties and their conjugation to oligonucleotides are found in, e.g., International Patent Publication WO 2019232255 and in Prakash et al., (2019).

In further examples, the delivery moiety includes an antibody, as described in, e.g., Dugal-Tessier et al., (2021), J Clin Med., 10(4):838.

Suitable examples of stabilizing moieties include, but are not limited to, polyethylene glycol (PEG), poly (oligo (ethylene glycol) methyl ether methacrylate) (POEGMA), and Poly(2-oxazoline)s (POx).

In some examples, where an ASO is linked to a functional moiety, the functional moiety is covalently linked to the ASO. In other examples, the functional moiety is non-covalently linked to the ASO.

Functional moieties can be linked to one or more of any nucleotides in an ASO at any of several positions on the sugar, base or phosphate group, as understood in the art and described in the literature, e.g., using a linker. Linkers can include a bivalent or trivalent branched linker. In some examples, the functional moiety is linked to the 5′ end of the ASO. In other examples, the functional moiety is linked to the 3′ end of the ASO.

In some examples compositions comprising any of the ASOs disclosed herein also include a delivery nanocarrier complexed with ASO. In some examples, a delivery nanocarrier is selected from among lipoplexes, liposomes, exosomes, inorganic nanoparticles, and DNA nanostructures. In other examples the delivery nanocarrier includes a lipid nanoparticle (LNP) encapsulating the ASO. In some examples the LNP comprises a neurotransmitter-derived lipidoid (NT-lipidoid) to facilitate delivery across the blood-brain barrier, as described in Ma et al., (2020). Various delivery ASO-nanocarrier complex formats are known in the art, as reviewed in, e.g., Roberts et al., supra.

### Pharmaceutical Compositions

Also provided herein are pharmaceutical compositions comprising any of the foregoing ASOs, non-viral expression vectors, modified messenger RNAs (mmRNAs), and viral expression vectors disclosed herein, and formulated with at least a pharmaceutically acceptable excipient, including a carrier, filler, preservative, adjuvant, solubilizer and/or diluent.

Pharmaceutical compositions containing any of the ASOs or expression vector compositions described herein, for use in the methods disclosed herein, can be prepared according to conventional techniques well known in the pharmaceutical industry and described in the published literature. In some examples, a pharmaceutical composition for treating a subject comprises a therapeutically effective amount of any ASO or expression vector disclosed herein.

Pharmaceutically acceptable salts are suitable for use in contact with the tissues of humans and lower animals without undue toxicity, irritation, allergic response, etc., and are commensurate with a reasonable benefit/risk ratio. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some examples, pharmaceutical compositions are formulated into any of a number of possible dosage routes or forms including, but not limited to, intravenous administration, intrathecal administration magna administration, tablets, capsules, gel capsules, liquid syrups, and soft gels. In some examples, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some examples, a pharmaceutical formulation disclosed herein is provided in a form including, but not limited to, a solution, emulsion, microemulsion, foam or liposome-containing formulation (e.g., cationic or noncationic liposomes).

In some examples, pharmaceutical formulations comprising any of the ASOs or expression vectors described herein may comprise one or more penetration enhancers, carriers, excipients or other active or inactive ingredients as appropriate and known to the skilled person. In some examples, where a pharmaceutical composition includes liposomes, such liposomes can also include sterically stabilized liposomes, e.g., liposomes comprising one or more specialized lipids. These specialized lipids result in liposomes with enhanced circulation lifetimes. In some examples, a sterically stabilized liposome comprises one or more glycolipids or is derivatized with one or more hydrophilic polymers, such as PEG moiety. In some examples, a surfactant is included in the pharmaceutical formulation.

In some examples, a pharmaceutical composition also includes a penetration enhancer to enhance the delivery of ASOs or non-viral expression vectors, e.g., to aid diffusion across cell membranes and/or enhance the permeability of a lipophilic drug.

In some examples, the penetration enhancers include a surfactant, a fatty acid, a bile salt, or a chelating agent.

In some examples, where administration is via a systemic route, e.g., intravenous, the method also includes a step to facilitate transfer of any of the ASOs or vectors described herein across the blood brain barrier (BBB) into the CNS, and especially into the brain. In some examples the BBB is transiently disrupted, e.g., by administration of one or more antibodies that disrupt Netrin-1 binding to Unc5B as described in Boyé et al., (2022).

In some examples, a pharmaceutical composition comprises a dose of ASOs or non-viral vectors ranging from about 0.01 mg/kg to 20 mg/kg, e.g., 0.05 mg/kg, 0.1 mg/kg, 0.2 mg/kg, 0.5 mg/kg, 1 mg/kg, 3 mg/kg, 5 mg/kg, 8 mg/kg, 10 mg/kg, 15 mg/kg, or another dose ranging from about 0.01 mg/kg to 20 mg/kg. In some examples, where an ASO disclosed herein is to be administered directly into the CNS or brain, e.g., by intracerebroventricular administration, the total dose ranges from about 50 mg to about 500 mg, e.g., 60 mg, 70 mg, 80 mg, 100 mg, 120 mg, 150 mg, 180 mg, 200 mg, 220 mg, 250 mg, 270 mg, 290 mg, 300 mg, 350 mg, 400 mg, 450 mg, or another dose from about 50 mg to about 500 mg. This dose range corresponds to approximately 0.050 mg/cm³ of brain volume to about 0.42 mg/cm³ of brain volume assuming an average human brain volume of about 1200 cm³.

In some examples, a pharmaceutical composition comprises multiple ASOs or AR expression vectors. In some examples, a pharmaceutical composition comprises, in addition to ASOs or AR expression vectors, another drug or therapeutic agent suitable for treatment of a subject suffering from a condition associated with OPA1 haploinsufficiency.

### Method of Treatment

According to a still further aspect of the invention, there is provided one or more antisense oligonucleotides as described herein for use in an antisense oligonucleotide-based therapy. Preferably, the therapy is for a disease related to OPA1 expression. More preferably, the therapy for a disease related to OPA1 expression is therapy for ADOA. Preferably the disease associated with OPA1 expression is associated with decreased levels of functional OPA1 protein expression. Preferably the decreased functional OPA1 is in patients who have ADOA, more preferably in ADOA where OPA1 haploinsufficiency is implicated.

More specifically, the antisense oligonucleotide may be selected from Table 1 and Table 2, or the group consisting of any one or more of SEQ ID Nos: 1-31; more preferably SEQ ID Nos: 1, 2, 3, 4 and 7; even more preferably SEQ ID Nos: 1, 3 and 4, and combinations or cocktails thereof. More specifically, the antisense oligonucleotide may be selected from Table 1, Table 2 and Table 4, or the group consisting of any one or more of SEQ ID Nos: 1-31 and 33-54; more preferably SEQ ID Nos: 1, 2, 3, 4, 7 and 33-44; even more preferably SEQ ID Nos: 1, 3, 4, 37, 40 and 44, and combinations or cocktails thereof. This includes sequences that can hybridise to such sequences under stringent hybridisation conditions, sequences complementary thereto, sequences containing modified bases, modified backbones, and functional truncations or extensions thereof that possess or modulate mRNA translation of an OPA1 gene transcript.

The invention extends also to a combination of two or more antisense oligonucleotides capable of binding to a selected target to induce splicing modulation in an OPA1 gene transcript. The combination may be a cocktail of two or more antisense oligonucleotides, a construct comprising two or more or two or more antisense oligonucleotides joined for use in an antisense oligonucleotide-based therapy.

There is therefore provided a method to treat, prevent or ameliorate the effects of a disease associated with OPA1 expression, comprising the step of:
a) administering to the patient an effective amount of one or more antisense oligonucleotides or pharmaceutical composition comprising one or more antisense oligonucleotides as described herein.

Preferably the disease associated with OPA1 expression in a patient is ADOA.

Therefore, the invention provides a method to treat, prevent or ameliorate the effects of ADOA, comprising the step of:
a) administering to the patient an effective amount of one or more antisense oligonucleotides or pharmaceutical composition comprising one or more antisense oligonucleotides as described herein.

Also provided herein is a method for increasing the OPA1 protein in a cell, the method comprising contacting the cell with a composition or pharmaceutical composition, as disclosed herein, whereby the amount of OPA1 protein in the cell is increased. Also provided herein is a method for increasing the level of OPA1 protein in a cell, ex vivo or in a tissue in vivo, the method comprising contacting the cell with an ASO or pharmaceutical composition, as disclosed herein, whereby the amount of OPA1 protein in the cell is increased.

Preferably, the therapy is used to increase the levels of functional OPA1 protein via a splicing modulation strategy. The increase in levels of OPA1 protein is preferably achieved by reducing the amount of OPA1 RNA containing aberrantly retained exon 7x and increasing the amount of OPA1 RNA that does not contain exon 7x.

In some examples, administration to a subject or contact with cells with any of the ASOs or pharmaceutical compositions disclosed herein increases the level of OPA1 protein about 1.1 to about 10-fold, e.g., 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the level in the tissue prior to the administration or contact. In one example, administration to a subject or contact with cells with any of the ASOs or pharmaceutical compositions disclosed herein increases the level of OPA1 protein about 1.1 to about 2.5-fold compared to the level in the tissue prior to the administration or contact.

Preferably administration of the antisense oligonucleotide results in the between 1.1 and 2.5-fold higher expression of the OPA1 protein than the expression of the OPA1 protein in subjects with symptomatic OPA1 mutations.

It will be apparent to the skilled person from the disclosure herein that antisense oligonucleotides of the disclosure that reduce expression of OPA1 gene transcript lacking exon 7x do so substantially without changing the proportion of other transcripts of the OPA1 gene. For example, antisense oligonucleotides of the disclosure reduce expression of OPA1 gene transcript lacking exon 7x without substantially affecting the relative expression levels of transcripts comprising exon 7 or lacking exon 7. In one example, the antisense oligonucleotides of the disclosure reduce expression of OPA1 gene transcript lacking exon 7x without reducing the expression level of transcripts lacking exon 7. The data herein suggest that antisense oligonucleotides of the disclosure reduce expression of OPA1 gene transcript lacking exon 7x without changing normal physiological ratios of transcripts of the OPA1 gene comprising exon 7 or lacking exon 7. Such antisense oligonucleotides are of interest therapeutically since transcripts lacking exon 7 are thought to increase the sensitivity of cells to apoptosis and, as a consequence, oligonucleotides that increase relative levels of transcripts lacking exon 7 are considered to be of less therapeutic benefit.

In one example, antisense oligonucleotides of the disclosure that reduce expression of OPA1 gene transcript lacking exon 7x do not affect the relative expression levels of OPA1 gene transcripts comprising exon 7 or lacking exon 7. In one example, antisense oligonucleotides of the disclosure that reduce expression of OPA1 gene transcript lacking exon 7x do not affect the relative expression levels of OPA1 gene transcripts comprising exon 7 or lacking exon 7 when administered to a cell compared to the level or ratio of the transcripts in a cell to which the antisense oligonucleotide has not been administered. For example, antisense oligonucleotides of the disclosure reduce expression of OPA1 gene transcript lacking exon 7x without changing normal physiological ratios of transcripts comprising exon 7 or lacking exon 7 when administered to a cell compared to the level or ratio of the transcripts in a cell to which the antisense oligonucleotide has not been administered. Normal physiological ratios of transcripts comprising exon 7 or lacking exon 7 will be apparent to the skilled person and/or disclosed herein. For example, normal physiological ratios of OPA1 gene transcripts comprising exon 7 or lacking exon 7 are approximately 1:4 of OPA1 gene transcript comprising exon 7 to OPA1 gene transcript lacking exon 7 (i.e., approximately 20% OPA1 gene transcript comprising exon 7 and approximately 80% OPA1 gene transcript lacking exon 7). Levels and/or ratios of the transcripts are determined using methods known in the art, such as, qPCR.

In one example, the relative expression levels of OPA1 gene transcripts comprising exon 7 or lacking exon 7 are at a ratio of approximately 20% OPA1 gene transcript comprising exon 7 and approximately 80% OPA1 gene transcript lacking exon 7. For example, the OPA1 gene transcripts comprising exon 7 or lacking exon 7 are at a ratio of between 15-25% OPA1 gene transcript comprising exon 7 and between 75-85% OPA1 gene transcript lacking exon 7.

In one example, the relative expression levels of OPA1 gene transcripts comprising exon 7 or lacking exon 7 are at a ratio of approximately 1:4 of OPA1 gene transcript comprising exon 7 to OPA1 gene transcript lacking exon 7.

The increase in functional OPA1 will preferably lead to a reduction in the quantity, duration or severity of the symptoms of an OPA1-related disease, such as ADOA.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease being treated, delaying the onset of that disease, or reducing the severity of its onset. “Treatment” or “prophylaxis” does not necessarily indicate complete eradication, cure, or prevention of the disease, or associated symptoms thereof.

The subject with the disease associated with OPA1 expression may be a mammal, including a human.

The antisense oligonucleotides of the present invention may also be used in conjunction with alternative therapies, such as drug therapies.

The present invention therefore provides a method of treating, preventing or ameliorating the effects of a disease associated with OPA1 expression, wherein the antisense oligonucleotides of the present invention and administered sequentially or concurrently with another alternative therapy associated with treating, preventing or ameliorating the effects of a disease associated with OPA1 expression. Preferably, the disease is ADOA.

### Delivery

The antisense oligonucleotides of the present invention also can be used as a prophylactic or therapeutic, which may be utilised for the purpose of treatment of a disease. Accordingly, in one embodiment the present invention provides antisense oligonucleotides that bind to a selected target in the OPA1 pre-mRNA to induce efficient and consistent correctly spliced OPA1 mRNA as described herein, in a therapeutically effective amount, admixed with a pharmaceutically acceptable carrier, diluent, or excipient.

There is also provided a pharmaceutical, prophylactic, or therapeutic composition to treat, prevent or ameliorate the effects of a disease related to OPA1 expression in a patient, the composition comprising:
a) one or more antisense oligonucleotides as described herein and
b) one or more pharmaceutically acceptable carriers and/or diluents.

Preferably, the antisense oligonucleotide of the present invention is delivered via a localised ocular route to avoid a systemic effect. Routes of administration include, but are not limited to, intravitreal, intracameral, subconjunctival, subtenon, retrobulbar, posterior juxtascleral, or topical (drops, eye washes, creams etc). Delivery methods include, for example, injection by a syringe and a drug delivery device, such as an implanted vitreal delivery device (i.e., VITRASERT®).

More preferably, the antisense oligonucleotide is administered via intravitreal injection at between 0.005-5 mg per eye, 0.005-1 mg per eye, 0.005-0.5 mg per eye, 0.01-5 mg per eye, 0.02-1 mg per eye, 0.01-0.5 mg per eye, 0.01-0.1 mg per eye, 0.01-0.5 mg per eye, 2-20 mg per eye, 0.5-20 mg per eye, or more preferably between 5-20 mg per eye. The antisense oligonucleotide may be administered via intravitreal injection at, for example, about 0.01 mg per eye, 0.02 mg per eye, 0.03 mg per eye, 0.04 mg per eye, 0.05 mg per eye, 0.06 mg per eye, 0.07 mg per eye, 0.08 mg per eye, 0.09 mg per eye, 0.1 mg per eye, 0.2 mg per eye, 0.3 mg per eye, 0.4 mg per eye, 0.5 mg per eye, 1 mg per eye, 2 mg per eye. Preferably, the antisense oligonucleotide is administered via intravitreal injection at about 0.01-0.5 mg per eye.

The antisense oligonucleotide may be administered at regular intervals for a short time period, e.g., daily for two weeks or less. However, in many cases the oligonucleotide is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

Dosing may be dependent on severity and responsiveness of the disease state to be treated, with the course of treatment lasting from several days to several months, or until a cure is effected or a diminution of the disease state is achieved. Alternatively, dosing may be titrated against disease progression rate. A baseline progression is established. Then the progression rate after an initial once off dose is monitored to check that there is a reduction in the rate. Preferably, there is no progression after dosing. Preferably, re-dosing is only necessary if progression rate is unchanged. Successful treatment preferably results in no further progression of the disease or even some recovery of vision. Optimal dosing schedules can be calculated from measurements of drug accumulation in the body of the patient. Persons of ordinary skill can easily determine optimum dosages, dosing methodologies and repetition rates.

Optimum dosages may vary depending on the relative potency of individual oligonucleotides and can generally be estimated based on EC50 values found to be effective in in vitro and in vivo animal models.

In general, dosage is administered via intravitreal injection at between 0.005-5 mg per eye and may be given once or more daily, weekly, monthly or yearly, or even once every 2 to 20 years. Repetition rates for dosing depend on progression rate of the disease. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, the dose may be administered via intravitreal injection at between 0.005-5 mg per eye, once or more daily, weekly, monthly or yearly, or even once every 2 to 20 years.

An effective in vivo treatment regimen using the antisense oligonucleotides of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligonucleotides of the invention may be determined from biological samples (tissue, blood, urine etc.) taken from a subject prior to, during and subsequent to administration of the antisense oligonucleotide. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

Intranuclear oligonucleotide delivery is a major challenge for antisense oligonucleotides. Different cell-penetrating peptides (CPP) localize PMOs to varying degrees in different conditions and cell lines, and novel CPPs have been evaluated by the inventors for their ability to deliver PMOs to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration and/or macromolecular translocation when administered intraocularly. CPPs are well-known in the art and are disclosed, for example in U.S. Application No. 2010/0016215, which is incorporated by reference in its entirety.

The present invention therefore provides antisense oligonucleotides of the present invention in combination with cell-penetrating peptides for manufacturing therapeutic pharmaceutical compositions.

### Excipients

The antisense oligonucleotides of the present invention are preferably delivered in a pharmaceutically acceptable composition. The composition may comprise about 1 nM to 1000 nM of each of the desired antisense oligonucleotide(s) of the invention. Preferably, the composition may comprise about 1 nM to 500 nM, 10 nM to 500 nM, 50 nM to 750 nM, 100 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the antisense oligonucleotide(s) of the invention.

The composition may comprise about 1 nM, 2 nM, 3 nM, 4 nM, 5 nM, 6 nM, 7 nM, 8 nM, 9 nM, 10 nM, 20 nM, 50 nM, 75 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM or 1000 nM of each of the desired antisense oligonucleotide(s) of the invention.

The present invention further provides one or more antisense oligonucleotides adapted to aid in the prophylactic or therapeutic treatment, prevention or amelioration of symptoms of a disease such as an OPA1 expression related disease or pathology in a form suitable for delivery to a patient.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Remington: The Science and Practice of Pharmacy, 22nd Ed., Pharmaceutical Press, PA (2013).

In a more specific form of the invention there are provided pharmaceutical compositions comprising therapeutically effective amounts of one or more antisense oligonucleotides of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hyaluronic acid may also be used. Such compositions may influence the physical state, stability, rate of in vivo release, and rate of in vivo clearance of the present proteins and derivatives. See, for example, Remington: The Science and Practice of Pharmacy, 22nd Ed., Pharmaceutical Press, PA (2013). The compositions may be prepared in liquid form, or may be in dried powder, such as a lyophilised form.

It will be appreciated that pharmaceutical compositions provided according to the present invention may be administered by any means known in the art. The pharmaceutical compositions for administration are administered by injection, orally, topically or by the pulmonary or nasal route. For example, the antisense oligonucleotides may be delivered by topical, intravenous, intra-arterial, intraperitoneal, intramuscular or subcutaneous routes of administration. The appropriate route may be determined by one of skill in the art, as appropriate to the condition of the subject under treatment. Preferably, the antisense oligonucleotides are delivered topically to the eye or by intraocular injection or intraocular implant, so that the effects on OPA1 production are spatially limited and are not systemic.

Formulations for topical administration include those in which the oligonucleotides of the disclosure are in admixture with a topical delivery agent such as hydrogels, lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, oligonucleotides of the disclosure may be encapsulated within liposomes or may form complexes thereto, in particular to cationic liposomes. Alternatively, oligonucleotides may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the antisense oligonucleotides of the disclosure can be delivered by topical or transdermal methods (e.g., via incorporation of the antisense oligonucleotides into, e.g., emulsions, with such antisense oligonucleotides optionally packaged into liposomes) including delivery to ocular surfaces. Such topical or transdermal and emulsion/liposome-mediated methods of delivery are described for delivery of antisense oligonucleotides in the art, e.g., in U.S. Pat. No. 6,965,025. Preferably the topical delivery is delivery to the eye.

The antisense oligonucleotides described herein may also be delivered via an implantable device. Design of such a device is an art-recognized process, with, e.g., synthetic implant design described in, e.g., U.S. Pat. No. 6,969,400. Preferably the implant is able to be implanted into the eye for sustained delivery of the antisense oligonucleotides.

Compositions and formulations for ocular administration, including ocular injection, topical ocular delivery and ocular implant may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of antisense oligonucleotides may be achieved by methods previously published. For example, delivery of the antisense oligonucleotide may be via a composition comprising an admixture of the antisense oligonucleotide and an effective amount of a block copolymer. An example of this method is described in US patent application US20040248833. Other methods of delivery of antisense oligonucleotides to the nucleus are described in Mann CJ et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

Antisense oligonucleotides can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like. It may be desirable to deliver the antisense oligonucleotide in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligonucleotide of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes that are believed to interact with negatively charged DNA molecules to form a stable complex. Liposomes that are pH-sensitive or negatively-charged are believed to entrap DNA or other oligonucleotide rather than complex with it. Both cationic and noncationic liposomes have been used to deliver DNA or other oligonucleotides to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

As known in the art, antisense oligonucleotides may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligonucleotide may also be combined with other pharmaceutically acceptable carriers or diluents to produce a pharmaceutical composition. Suitable carriers and diluents include isotonic saline solutions, for example phosphate-buffered saline. The composition may be formulated for topical, parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992)256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The antisense oligonucleotides of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention, i.e. salts that retain the desired biological activity of the parent compound and do not impart undesired toxicological effects thereto. For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be via topical (including ophthalmic and mucous membranes, as well as rectal delivery), pulmonary (e.g., by inhalation or insufflation of powders or aerosols, including by nebulizer, intratracheal, intranasal, epidermal and transdermal), oral or parenteral routes. Parenteral administration includes intravenous, intra-arterial, subcutaneous, intraperitoneal, intraocular or intramuscular injection or infusion; or intracranial, e.g., intrathecal or intraventricular, administration. Oligonucleotides with at least one 2′-O-methoxyethyl modification are believed to be particularly useful for intraocular administration. Preferably, the antisense oligonucleotide is delivered via the intraocular route.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

### Use

According to another aspect of the invention there is provided the use of one or more antisense oligonucleotides as described herein in the manufacture of a medicament for the modulation or control of a disease associated with OPA1 protein expression.

The invention also provides for the use of purified and isolated antisense oligonucleotides as described herein, for the manufacture of a medicament for treatment of a disease associated with OPA1 protein expression.

There is also provided the use of purified and isolated antisense oligonucleotides as described herein, for the manufacture of a medicament to treat, prevent or ameliorate the effects of a disease associated with OPA1 protein expression.

Preferably, the OPA1-related disease is ADOA. Preferably the disease associated with OPA1 protein expression is associated with decreased levels of functional OPA1 protein. Preferably the decreased levels of functional OPA1 protein are in patients who have ADOA, more preferably in ADOA where OPA1 haploinsufficiency is implicated.

The invention extends, according to a still further aspect thereof, to cDNA or cloned copies of the antisense oligonucleotide sequences of the invention, as well as to vectors containing the antisense oligonucleotide sequences of the invention. The invention extends further also to cells containing such sequences and/or vectors.

### Kits

There is also provided a kit to treat, prevent or ameliorate the effects of a disease associated with OPA1 protein expression in a patient, which kit comprises at least an antisense oligonucleotide as described herein and combinations or cocktails thereof, packaged in a suitable container, together with instructions for its use. Preferably the disease associated with OPA1 protein expression is ADOA.

In a preferred embodiment, the kits will contain at least one antisense oligonucleotide as described herein or as shown in Table 1 and Table 2. Preferably, the antisense oligonucleotide is selected from the list comprising: SEQ ID Nos: 1-31; more preferably SEQ ID Nos: 1, 2, 3, 4 and 7; even more preferably SEQ ID Nos: 1, 3 and 4.

In a preferred embodiment, the kits will contain at least one antisense oligonucleotide as described herein or as shown in Table 1, Table 2 and Table 4.Preferably, the antisense oligonucleotide is selected from the list comprising: SEQ ID Nos: 1-31 and 33-54; more preferably SEQ ID Nos: 1, 2, 3, 4, 7 and 33-44; even more preferably SEQ ID Nos: 1, 3, 4, 37, 40 and 44.

In one embodiment, the kits will contain a cocktail of antisense oligonucleotides, as described herein.

The kits may also contain peripheral reagents such as buffers, stabilizers, etc.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

In an embodiment, the kit of the present invention comprises a composition comprising a therapeutically effective amount of an antisense oligonucleotide capable of binding to a selected target on an OPA1 gene transcript to modify translation in an OPA1 gene transcript or part thereof. In an alternative embodiment, the formulation is in pre-measured, pre-mixed and/or pre-packaged. Preferably, the intraocular solution is sterile.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the formulations of the present invention. In one embodiment, the instructions indicate that the formulation of the present invention is suitable for the treatment of ADOA. Such instructions may also include instructions on dosage, as well as instructions for administration via topical delivery to the eye or via intraocular injection.

The antisense oligonucleotides and suitable excipients can be packaged individually so to allow a practitioner or user to formulate the components into a pharmaceutically acceptable composition as needed. Alternatively, the antisense oligonucleotides and suitable excipients can be packaged together, thereby requiring de minimis formulation by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents. Sequence identity numbers (“SEQ ID NO:”) containing nucleotide and amino acid sequence information included in this specification are collected at the end of the description and have been prepared using the program Patentln Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense oligonucleotide nomenclature system was proposed and published to distinguish between the different antisense oligonucleotides (see Mann et al., (2002) J Gen Med 4, 644-654). This nomenclature became especially relevant when testing several slightly different antisense oligonucleotides, all directed at the same target region,

$$H \# A / D ( x : y )$$

the first letter designates the species (e.g. H: human, M: murine)
“#” designates target exon number
“A/D” indicates acceptor or donor splice site at the beginning and end of the exon, respectively
(x y) represents the annealing coordinates where “−” or “+” indicate intronic or exonic sequences respectively. As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense oligonucleotide. Entirely exonic annealing coordinates that would be represented by A(+65+85), that is the site between the 65th and 85th nucleotide, inclusive, from the start of that exon.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

### Example 1

### Identification of OPA1 Target Sequences and ASO Design

The region of OPA1 intron 7 and exon is shown in FIG. 1A. Antisense oligonucleotides (ASOs) 24-25 nucleotides in length (Table 1, FIG. 1B) and 17 nucleotides in length (Table 2) were designed to target the intronic splice enhancer motifs in intron 7 to mediate exclusion of exon 7x and generate productive OPA1 transcripts. Further refinement of ASO sequences demonstrating protein upregulation was performed by micro-walk or engineered mismatch oligos and re-validated by ddPCR and protein assays using the protocols described in Example 2 below.

### Example 2

### Screening of Phosphorodiamidate Morpholino Oligonucleotides (PMOs) targeting Intron 7 of OPA1 in Autosomal Dominant Optic Atrophy (ADOA) Patient Cells

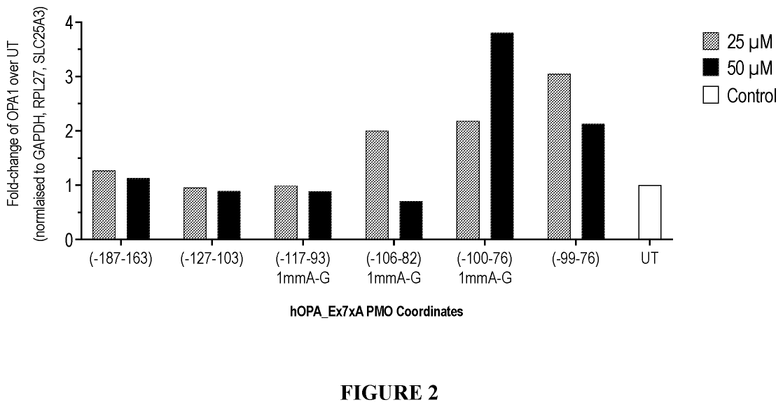

The above-identified ASO sequences 24 and 25 nucleotides in length were synthesized as PMOs. Antisense PMOs targeting intron 7 of OPA1 (as described in Example 1) were nucleofected into ADOA patient fibroblasts carrying the OPA1 mutation (c.2708_2711delTTAG) using the NEONR electroporation system (ThermoFisher) and the nucleofected cells were cultured for 5 days. Total RNA was extracted using the MagMAX™-96 Total RNA Isolation kit and the level of OPA1 transcript was assessed by digital droplet PCR (Qiagen; probe catalog number: dHsaCPE5043545). OPA1 transcript expression was normalized to GAPDH. RPL27 and SCL25A3 transcript levels (Qiagen; probe catalog number: dHsaCPE5031596, dHsaCPE5036407, dHsaCPE5032926 respectively). Three PMOs upregulated OPA1 transcript expression compared to untreated control at 48 hr post-treatment. The PMO hOPA1_Ex7xA(−100−76)1 mmA-G exhibited the highest OPA1 transcript upregulation; up to 3.5-fold at 48 hr as compared to untreated patient fibroblasts (n=1 biological replicate) (FIG. 2).

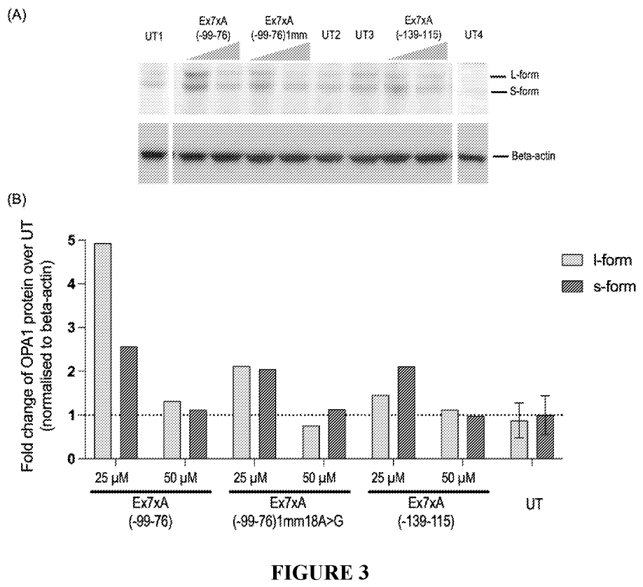

In addition to OPA1 transcript assessment, total protein was harvested (48 hr) from the transfected cells using the CytoBuster protein extraction reagent (Merck Millipore) following the manufacturer's instruction and assessed by western blot assay using rabbit anti-OPA1 monoclonal antibody (Cell Signaling, catalogue number 67589) at a dilution of 1:250 in 5% BSA in TBST buffer followed by goat anti-rabbit IgG H&L antibody (Abcam, catalogue number ab216773, IRDye® 800CW). Beta-actin served as loading control and was detected using monoclonal mouse anti-beta actin antibody (Sigma-Aldrich, catalogue number A5441) followed by goat anti-mouse IgG H&L antibody (Abcam, catalogue number ab216776, IRDye® 680RD). Expression levels of OPA1 protein were compared between no ASO-transfected cells (UT) and OPA1 NMD ASO-transfected cells. The PMO hOPA1_Ex7xA(−99−76) exhibited upregulation of OPA1 expression; up to 5-fold at 48 hr as compared to untreated patient fibroblasts (FIG. 3).

### Example 3

### Screening of Cell Penetrating Peptide-Conjugated Phosphorodiamidate Morpholino Oligonucleotides (PPMOs) Targeting the Intron 7 of OPA1 in Autosomal Dominant Optic Atrophy (ADOA) Patient Cells

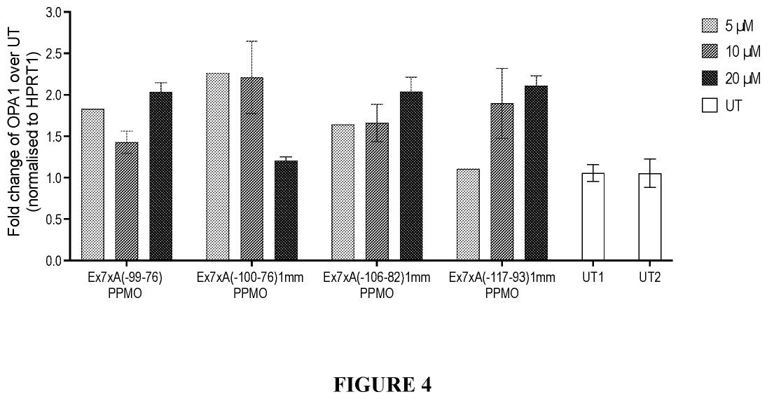

Selected OPA1 PMOs were conjugated to a cell penetrating peptide (PPMOs). PPMOs were incubated to ADOA patient fibroblasts carrying the OPA1 mutation (c.985-1G>A) and the transfected cells were cultured for 5 days. Total RNA was extracted using the MagMAX™-96 Total RNA Isolation kit and the level of OPA1 transcript was assessed by digital droplet PCR (Qiagen; probe catalog number: dHsaCPE5043545). OPA1 transcript expression was normalized to HPRT1 (Qiagen; probe catalog number: dHsaCPE5192872). The majority of PMOs upregulated OPA1 transcript expression compared to untreated control at 5 days post-treatment. The PMO hOPA1_Ex7xA(−100−76)1 mmA-G showed the highest upregulation of OPA1 transcript levels; up to 2.2-fold at 5 days as compared to untreated patient fibroblasts (n=1 biological replicate) (FIG. 4).

### Example 4

### Refinement of PMO Sequences Targeting Intron 7 of OPA1 in ADOA Patient Cells

PMOs were redesigned to microwalk around the parental sequence identified in exon Examples 2 and 3. FIG. 4 illustrates the binding sites of refined ASOs (ASOs listed in Table 4) on the OPA1 target portion in relation to parental sequences in Table 1. Mismatched base substation and oligonucleotide lengthening were employed to improve the efficacy of OPA1 upregulation.

### Example 5

### Screening of PMO Refinement Sequences in ADOA Patient Derived Fibroblasts

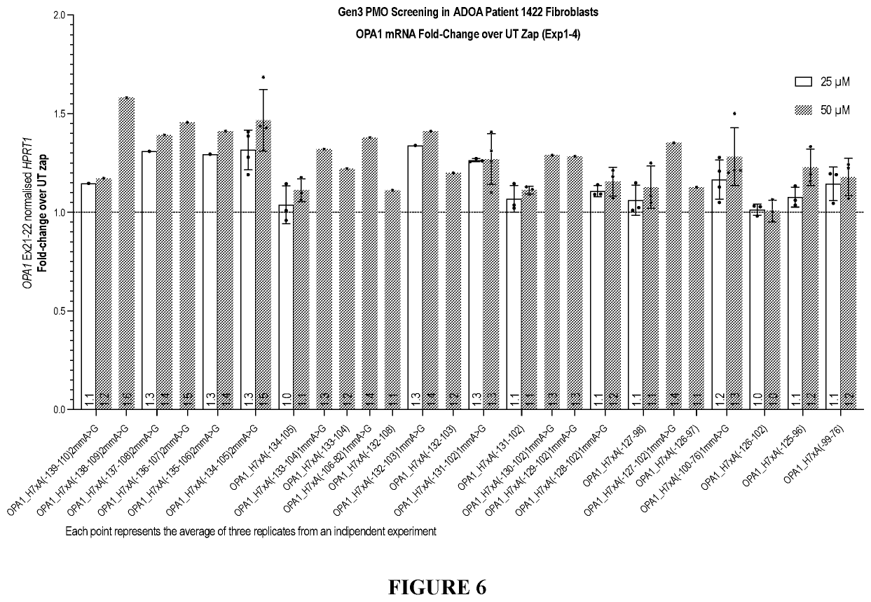

PMOs with refinement sequences were transfected using the NEONR electroporation system (ThermoFisher) to ADOA patient fibroblasts carrying the OPA1 mutation (c.985-1G>A) and were incubated for 48 hr. Total RNA was extracted using the MagMAX™-96 Total RNA Isolation kit and the level of OPA1 transcript was assessed by digital droplet PCR (Qiagen; probe catalog number: dHsaCPE5043545). OPA1 transcript expression was normalized to the HPRT1 level (Qiagen; probe catalog number: dHsaCPE5192872). The majority of refinement PMOs upregulated OPA1 transcript expression compared to untreated control at 48 hr post-treatment (n=1-4 biological replicates) (FIG. 6).

### Example 6

### PMO Treatment Induces OPA1 Protein Upregulation in ADOA Fibroblasts

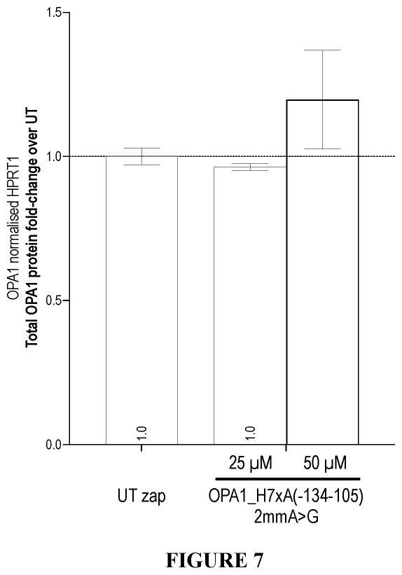

Selected refinement PMO was transfected in triplicate using the NEONR electroporation system (ThermoFisher) to ADOA patient fibroblasts carrying the OPA1 mutation (c.2708_2711delTTAG) and incubated for 120 hr. Total protein was harvested from the transfected cells using RIPA buffer (ThermoFisher) following the manufacturer's instruction and assessed by western blot assay using rabbit anti-OPA1 monoclonal antibody (Cell Signaling, catalogue number 67589) at a dilution of 1:250 in 5% BSA in TBST buffer followed by goat anti-rabbit IgG H&L antibody (Abcam, catalogue number ab216773, IRDye® 800CW). HPRT1 served as the loading control and was detected using HPRT1 Polyclonal antibody (ProteinTech, catalogue number 15059-1-AP). Expression levels of OPA1 protein were compared between no ASO-transfected cells (UT) and OPA1 NMD ASO-transfected cells. FIG. 7 shows the PMO exhibited upregulation of OPA1 expression compared to untreated patient fibroblasts (n=1 biological replicate).

### Example 7

### PMO Mediated OPA1 Upregulation and Maintaining the Balance of OPA1 Isoforms

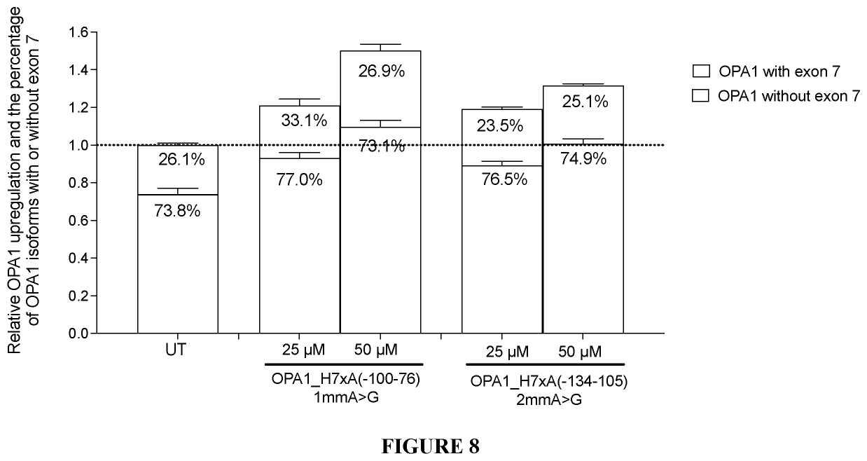

Selected parental and refinement PMOs were screened for the efficacy for total OPA1 upregulation and the expression of OPA1 isoforms both with and without exon 7. PMOs were transfected to ADOA patient fibroblasts carrying the OPA1 mutation (c.2708_2711delTTAG) and incubated for 48 hr. Total RNA was extracted using the MagMAX™-96 Total RNA Isolation kit and the level of OPA1 transcript was assessed by digital droplet PCR (Qiagen; probe catalog number: dHsaCPE5043545). OPA1 transcript expression was normalized to HPRT1 (Qiagen; probe catalog number: dHsaCPE5192872). OPA1 isoform expression was analysed by RT-PCR to determine the proportion of OPA1 isoforms with and without exon 7. FIG. 8 shows the PMO mediated total OPA1 upregulation while maintaining the proportion of OPA1 isoforms.

### Example 8

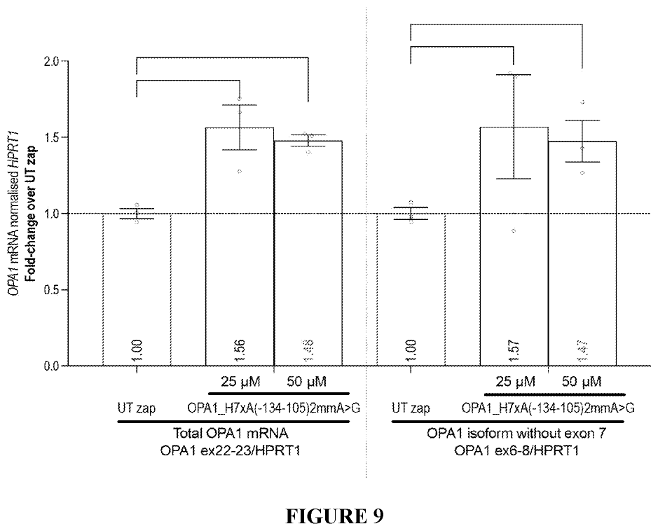

Quantification of OPA1 mRNA isoforms shows correlation with total OPA1 levels. PMO OPA1_Ex7xA(−134−105)2 mmA>G (25 and 50 μM) was incubated to patient derived fibroblast carrying the OPA1 mutation (c.2708_2711delTTAG) and samples were harvested for quantification of total and OPA1 isoform upregulation using a ddPCR assay. Primers targeting exons 22-23 and exons 6-8 were used to determine the levels of total OPA1 mRNA and OPA1 isoforms without exon 7, respectively and normalised to HPRT1 levels. As shown in FIG. 9, the expression of OPA1 isoforms without exon 7 increased by 1.5-1.6 fold correlated with the levels of total OPA1 mRNA confirming the ability of the PMO to increase OPA1 upregulation while maintain the balance of OPA1 isoforms.

### Example 9

### Peptide-Conjugated PMO (PPMO) Treatment Induces Total OPA1 mRNA in RGC Enriched Culture Derived from an ADOA Patient

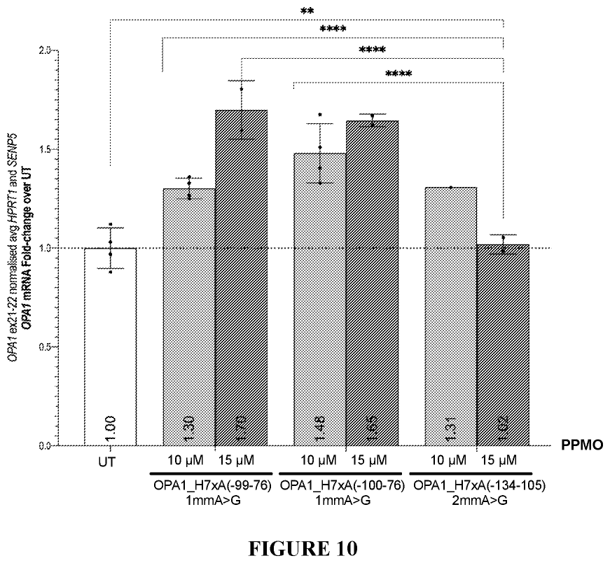

PMO hOPA1_Ex7xA(−100−76)1 mmA-G was conjugated with a cell penetrating peptide and was incubated to iPSC-RGCs derived from an ADOA patient carrying the OPA1 mutation (c.985-1G>A) for 120 hr in triplicates. Total RNA was extracted using the MagMAX™-96. Total RNA Isolation kit and the level of OPA1 transcript was assessed by digital droplet PCR (Qiagen; probe catalog number: dHsaCPE5043545). OPA1 transcript expression was normalized to the HPRT1 level (Qiagen; probe catalog number: dHsaCPE5192872). FIG. 10 shows the PMO hOPA1_Ex7xA(−100−76)1 mmA-G mediated upregulation of total OPA1 transcript by up to 1.7-fold at 15 μM as compared to untreated patient fibroblasts.

1. An isolated or purified antisense oligonucleotide that binds to intron 7 of a OPA1 gene pre-mRNA.

2. The isolated or purified antisense oligonucleotide of claim 1 that:
a) induces increased production of functional OPA1 protein or part thereof;
b) increases translation of functional OPA1 protein through exclusion of a nonsense-mediated RNA decay-inducing exon; and/or
c) increases translation of functional OPA1 protein through exclusion of nonsense-mediated RNA decay-inducing (NMD) exon 7x.

3. The isolated or purified antisense oligonucleotide of claim 1, wherein the antisense oligonucleotide is:
(i) a phosphorodiamidate morpholino oligonucleotide; and/or
(ii) selected from the group consisting of:
a) Table 1, Table 3 and Table 4;
b) SEQ ID Nos: 1-31;
c) SEQ ID Nos: 33-54;
d) SEQ ID Nos: 1, 2, 3, 4, 7 and 33-44;
e) SEQ ID Nos: 1, 3, 4, 37, 40 and 44; and
f) combinations or cocktails thereof.

4. (canceled)

5. The isolated or purified antisense oligonucleotide of claim 1, wherein the antisense oligonucleotide does not alter the relative expression levels or normal physiological ratios of OPA1 gene transcripts comprising exon 7 or lacking exon 7 when administered to a cell compared to the level or ratios of the transcripts in a cell to which the antisense oligonucleotide has not been administered.

6. A method for manipulating translation of the OPA1 gene transcript, the method comprising the step of:
i. providing one or more of the antisense oligonucleotides according to claim 1 and allowing the oligonucleotide(s) to bind to a target nucleic acid site.

7. A method to treat, prevent or ameliorate the effects of a disease associated with OPA1 expression, the method comprising the step of:
i. administering to the patient an effective amount of one or more antisense oligonucleotides according to claim 1.

8-10. (canceled)

11. The method of claim 7, wherein the disease associated with OPA1 expression is:
a) associated with decreased levels of functional OPA1 protein expression;
b) in a patient with Autosomal Dominant Optic Atrophy (ADOA);
c) associated with decreased levels of functional OPA1 protein expression in patients who have ADOA; and/or
d) associated with decreased levels of functional OPA1 protein expression in patients who have ADOA where OPA1 haploinsufficiency is implicated.

12. The antisense oligonucleotide according to claim 1, wherein the antisense oligonucleotide comprises a backbone modification.

13. The antisense oligonucleotide according to claim 12, wherein the antisense oligonucleotide comprises a backbone modification comprising a phosphorothioate linkage or a phosphorodiamidate linkage.

14. The antisense oligonucleotide according to claim 12, wherein the antisense oligonucleotide comprises a phosphorodiamidate morpholino, a locked nucleic acid, a peptide nucleic acid, a 2′-O-methyl, a 2′-Fluoro, or a 2′-O-methoxyethyl moiety.

15. The antisense oligonucleotide according to claim 12, wherein the antisense oligonucleotide comprises at least one modified sugar moiety, wherein each sugar moiety in the antisense oligonucleotide is a modified sugar moiety.

16. (canceled)

17. The antisense oligonucleotide according to claim 12, wherein the antisense oligonucleotide comprises a 2′-O-methoxyethyl moiety, wherein each nucleotide of the antisense oligonucleotide comprises a 2′-O-methoxyethyl moiety.

18. (canceled)

19. A vector for expression, in a mammalian cell, of an antisense RNA (AR) according to claim 1, wherein the expression vector comprises a cell type-selective or tissue-selective promoter for driving expression of the antisense RNA in the mammalian cell and/or an inducible promoter.

20. (canceled)

21. The vector according to claim 19, wherein the cell type-selective or tissue selective promoter is selective for expression in a cell type or tissue selected from the list consisting of: an ocular tissue, retinal ganglion cells, neuronal cells, glial cells, astrocytes, and photoreceptors.

22. (canceled)

23. The vector according to claim 19, wherein the vector is a non-viral vector or a viral vector, wherein the viral vector is a recombinant virus selected from the group consisting of: adeno-associated virus (AAV), adenovirus, lentivirus, and anellovirus.

24. (canceled)

25. (canceled)

26. The antisense oligonucleotide or vector according to claim 1, wherein the nucleotide sequence of the antisense oligonucleotide or the antisense RNA consists of 10 to 50 nucleotides, 15 to 40 nucleotides, 18 to 40 nucleotides, 17 to 25 nucleotides, 20 to 35 nucleotides, 20 to 30 nucleotides, 22 to 30 nucleotides, 24 to 30nucleotides, 25 to 30 nucleotides, or 26 to 30 nucleotides.

27. (canceled)

28. The antisense oligonucleotide according to claim 26, wherein the antisense oligonucleotide comprises one or more phosphorodiamidate morpholino moieties.

29. The antisense oligonucleotide according to claim 1, wherein the antisense oligonucleotide further comprises a linked functional moiety, wherein the functional moiety comprises a delivery moiety or a stabilising moiety.

30. (canceled)

31. The antisense oligonucleotide according to claim 29, wherein the delivery moiety:
(a) is selected from the group consisting of lipids, peptides, carbohydrates, and antibodies; or
(b) comprises a cell-penetrating peptide (CPP); or
(c) comprises a N-acetylgalactosamine (GalNAc) moiety.

32-34. (canceled)

35. The antisense oligonucleotide according to claim 29, wherein the functional moiety is covalently linked to the antisense oligonucleotide or is non-covalently linked to the antisense oligonucleotide, wherein the functional moiety is linked to the 5′ end of the antisense oligonucleotide or is linked to the 3′ end of the antisense oligonucleotide.

36-38. (canceled)
